(19) United States
(12) Patent Application Publication
(10) Pub. No.: US 2014/0010762 A1
(43) Pub. Date: Jan. 9, 2014

(54) IMAGING AGENTS

(71) Applicant: Margaret Struthers Drummond, Standlake (GB)

(72) Inventors: Michael Hugh Charlton, Abingdon (GB); David Festus Charles Moffat, Abingdon (GB); Stephen John Davies, Abingdon (GB); Alan Hastings Drummond, Abingdon (GB)

(73) Assignee: Chroma Therapeutics Ltd, Oxfordshire (GB)

(21) Appl. No.: 13/994,835

(22) PCT Filed: Dec. 16, 2011
(86) PCT No.: PCT/GB11/01729

§ 371 Date: Sep. 26, 2013

(30) Foreign Application Priority Data
Dec. 17, 2010 (GB) 1021467.4

(51) Int. Cl. A61K 49/00; C07F 5/02; G01N 33/58; C07D 271/12

(52) U.S. Cl. CPC A61K 49/0021; C07D 271/12; C07F 5/02; G01N 33/582
USPC 424/9.6; 548/126; 544/229; 435/29

(57) ABSTRACT

An imaging agent for cells which produces an intracellular imaging signal proportional to the amount of hCE-1 in the cells independently of the amount of hCE-2 and/or hCE-3 in the cells, said imaging agent being a covalent conjugate of (a) an imaging agent and (b) an alpha mono- or di-substituted amino acid ester, wherein (a) is directly linked to (b), or (a) is indirectly linked to (b) by a linker radical, and wherein said direct or indirect linkage is via the amino group of (b), and wherein the amino group is not directly linked to a carbonyl group, and wherein the said alpha mono- or di-substituted amino acid ester part is selectively hydrolysable to the corresponding carboxylic acid part by the intracellular carboxylesterase enzyme hCE-1 relative to the intracellular enzymes hCE-2 or hCE-3.

This invention relates to imaging agents which produce an intracellular imaging signal proportional to the amount of the intracellular carboxy esterase hCE-1 in the cells independently of the amount of the intracellular carboxy esterase hCE-2 and/or hCE-3 in the cells. They contain an alpha amino acid ester motif either directly or indirectly linked by a linker radical to the rest of the agent, that ester motif being one which is more rapidly and/or completely hydrolysed to the corresponding acid by hCE1 relative to hCE-2 and hCE-3. The imaging agents having the amino acid ester motifs easily pass into the cells, but the hydrolysis products, namely the acids, do not easily pass out of the cells. Hence the imaging signal is more intense in cells which accumulate the acid hydrolysis product. Since monocytic cells, such as macrophages, contain hCE-1 but other cell types in general do not, or contain only insignificant amounts of hCE-1, the signal from the imaging agents of the invention in such cells is more intense than that in other cell types, and the imaging agents of the invention are therefore useful as selective imaging agents for monocytes.

### BACKGROUND TO THE INVENTION

Certain medical diagnostic methods enable the investigation or visualisation of tissues or cells of interest in the human or animal body without resorting to surgical or other invasive techniques. One such branch of medical diagnostics relates to imaging agents, their administration to the body, and the collection and analysis of the resulting imaging data.

Imaging agents are chemical entities having a detectable group which gives rise to a signal which is detected by means external to the body. An imaging signal is one actively arising from a detectable emission from the imaging agent itself, or passively detectable as a result of stimulation of the imaging agent by external means. Once an imaging agent is administered to the body, its distribution throughout the tissues, cells and internal cavities of the body may be monitored by a detector. The detector gathers information which is used to generate an image, thereby providing valuable information about the internal environment of the body. The person skilled in the art knows of numerous imaging techniques involving imaging agents and external detection means.

One well known branch of imaging techniques relies on imaging agents labelled with one or more specific radioisotopes. The imaging technique known as single photon emission computed tomography (hereinafter referred to as SPECT) is used for detecting the radiation emitted from certain decaying radioisotopes such as ⁹⁹Tc, ¹²³I, and ²⁰¹Tl. An alternative technique known as positron emission tomography (hereinafter referred to as PET) is used for detecting radiation emitted from certain decaying radioisotopes such as ¹¹C, ¹³N, ¹⁸F and ⁶⁴Cu.

Fluorescent imaging agents, which have a fluorescent detectable group, are also well known. The radiation emitted from the fluorescent group is detected and used to generate an image showing the distribution of the imaging agent.

X-ray imaging agents, also known as contrast agents, have a detectable group which attenuates an X-ray beam. By administering the imaging agent, and taking an X-ray image of the target area, it is possible to obtain an image showing the concentration of the imaging agent in that area. It is known in the art that elements having a high number of electrons per atom of the element (corresponding to a high atomic weight) are especially, effective at attenuating X-rays. Iodine is well known as a strong X-ray attenuator, and is therefore commonly used in the detectable group in X-ray contrast agents.

Imaging agents may be used in the technique known as magnetic resonance imaging (hereinafter referred to as MRI). MRI imaging agents, also known as contrast agents, alter certain properties of molecules in the vicinity of the imaging agent, thus enabling a detector to distinguish the areas in which the imaging agents have accumulated. Information gathered from the detector enables the generation of an image providing improved visibility of internal body structure. MRI imaging agents are detectable because they alter the relaxation times of protons in tissues and body cavities in the immediate vicinity of the agents. The most commonly used imaging agents for MRI contrast enhancement are gadolinium-based, particularly gadolinium-containing macrocycles.

There is a continuing need for improved imaging agents which enable more detailed visualisation of the cells and tissues of the human or animal body. In particular, there is a need for imaging agents which may be targeted at specific tissues and or cells. Various approaches to cell targeting are known and include pH driven nanomolecules (Org. Lett. 2006, 8, 3363), nanoparticle enhanced imaging (Cancer Biomarkers 2009, 5, 59), Antibody Directed Prodrug Enzyme Therapy ADEPT (Clin Cancer Res. 2000 6, 765-72), antibody-drug conjugation (Curr Opin Chem Biol. 2010, 14, 529-37).

In particular, there is a need for imaging agents which allow selective imaging of monocytic cells, in particular macrophages. Non-invasive imaging of such cells is essential for in vivo identification of sites of inflammatory cell foci in the progression of diseases such as atherosclerosis, COPD and rheumatoid arthritis.

### BRIEF DESCRIPTION OF THE INVENTION

This invention makes available imaging agents which produce an intracellular imaging signal proportional to the amount of the intracellular carboxy esterase hCE1 in the cells independently of the amount of the intracellular carboxy esterase hCE-2 and/or hCE-3 in the cells.

It takes advantage of the fact that lipophilic (low polarity or charge neutral) molecules pass through the cell membrane and enter cells relatively easily, and hydrophilic (higher polarity, charged) molecules do not. Hence, if a lipophilic motif is attached to a given imaging agent, allowing the agent to enter the cell, and if that motif is converted in the cell to one of higher polarity, it is to be expected that the agent with the higher polarity motif attached would accumulate within the cell. The accumulation of imaging agent with the higher polarity motif attached is therefore expected to result in increased concentration and prolonged residence in the cell.

The present invention makes use of the fact that there are carboxylesterase enzymes within cells, which may be utilised to hydrolyse an alpha amino acid ester motif attached to a given imaging agent to the parent acid. Therefore, an imaging agent may be covalently linked to an alpha amino acid ester and administered, in which form it readily enters the cell where it is hydrolysed efficiently by one or more intracellular carboxylesterases, and the resultant alpha amino acid imaging agent conjugate accumulates within the cell, thus allowing selective detection and imaging of that cell-type. It has also been found that by modification of the alpha amino acid motif, or the way in which it is covalently linked, imaging agents can be targeted to monocytes and macrophages. Herein, unless “monocyte” or “monocytes” is specified, the term macrophage or macrophages will be used to denote macrophages (including tumour associated macrophages) and/or monocytes.

### DETAILED DESCRIPTION OF THE INVENTION

The present invention makes available an imaging agent for cells which produces an intracellular imaging signal proportional to the amount of hCE-1 in the cells independently of the amount of hCE-2 and/or hCE-3 in the cells, said imaging agent being a covalent conjugate of (a) an imaging agent and (b) an alpha mono- or di-substituted amino acid ester, wherein

(a) is directly linked to (b), or (a) is indirectly linked to (b) by a linker radical, and wherein said direct or indirect linkage is via the amino group of (b), and wherein
the amino group is not directly linked to a carbonyl group, and wherein
the said alpha mono- or di-substituted amino acid ester part is selectively hydrolysable to the corresponding carboxylic acid part by the intracellular carboxylesterase enzyme hCE-1 relative to the intracellular enzymes hCE-2 or hCE-3.

As stated, the invention is concerned with the modification of imaging agents. The invention is of general application, not restricted by the chemical identity of the imaging agent.

The alpha amino acid ester motif obviously must be a substrate for the carboxylesterase if the former is to be hydrolysed by the latter within the cell. Intracellular carboxylesterases are rather promiscuous in general, in that their ability to hydrolyse does not depend on very strict structural requirements of the amino acid ester substrate. Hence most methods of covalently linking the alpha amino acid ester motif to an imaging agent will allow hydrolysis.

Intracellular carboxylesterase enzymes capable of hydrolysing the ester group of a conjugated alpha amino acid ester to the corresponding acid include the three known human carboxylesterase (“hCE”) enzyme isotypes hCE-1 (also known as CES-1), hCE-2 (also known as CES-2) and hCE-3 (Drug Disc. Today 2005, 10, 313-325). Although these are considered to be the main enzymes other carboxylesterase enzymes such as biphenylhydrolase (BPH) may also have a role in hydrolysing the conjugates.

The broken cell assay described below is a simple method of confirming that a given conjugate of imaging agent and alpha amino acid ester, or a given alpha amino acid ester to be assessed as a possible carboxylesterase ester motif, is hydrolysed as required. These enzymes can also be readily expressed using recombinant techniques, and the recombinant enzymes may be used to determine or confirm that hydrolysis occurs.

It is a feature of the invention that the desired conjugate retains the covalently linked alpha amino acid motif when hydrolysed by the carboxylesterase within the cell, since it is the polar carboxyl group of that motif which prevents or reduces clearance of the hydrolysed conjugate from the cell, and thereby contributes to its accumulation within the cell. Since cells in general contain several types of peptidase enzymes, it is preferable that the conjugate, or more especially the hydrolysed conjugate (the corresponding acid), is not a substrate for such peptidases. In particular, it is strongly preferred that the alpha amino acid ester group should not be the C-terminal element of a dipeptide motif in the conjugate. Furthermore, as explained later, in order to achieve macrophage selectivity it is essential that the amino group of the alpha amino acid ester motif is not linked directly to a carbonyl group. However, apart from these limitations on the mode of covalent attachment, the alpha amino acid ester group may be covalently attached to the imaging agent via its amino group. In some cases the imaging agent will have a convenient point of attachment for the alpha amino acid ester motif, and in other cases a synthetic strategy will have to be devised for its attachment.

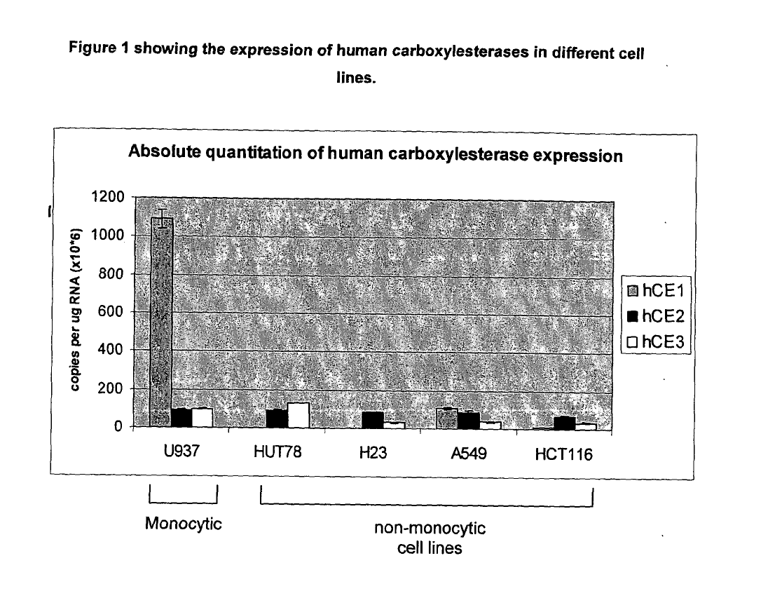

It has been found that cells that only express the carboxylesterases hCE-2, and/or hCE-3 and recombinant forms of these enzymes will only hydrolyse amino acid ester conjugates to their resultant acids if the nitrogen of the alpha amino acid group is either unsubstituted or is directly linked to a carbonyl group, whereas cells containing hCE-1, or recombinant hCE-1 can hydrolyse amino acid conjugates with a wide range of groups on the nitrogen. This selectivity requirement of hCE-2 and hCE-3 can be turned to advantage where it is required that the modulator should target enzymes or receptors in certain cell types only. It has been found that the relative amounts of these three carboxylesterase enzymes vary between cell types (see FIG. 1 and database at http://symatlas.gnf.org/SymAtlas (note that in this database hCE3/CES3 is referred to by the symbol FLJ21736)). If the modulator is intended to act only in cell types where hCE-1 is found, attachment of an alpha amino acid ester motif wherein the amino group is directly linked to a group other than carbonyl results in the hydrolysed modulator conjugate accumulating preferentially in cells with effective concentrations of hCE-1. Stated in another way, specific accumulation of the acid derived from the imaging agent conjugate in hCE-1 expressing cells can be achieved by linking the alpha amino acid ester motif to the imaging agent via the amino group of the amino acid ester part, but not such that the nitrogen of the amino group is directly linked to a carbonyl group (for example linkage via an amide bond —NH—C(═O)— is not allowed).

Macrophages are known to play a key role in inflammatory disorders through the release of cytokines in particular TNFα and IL-1 (van Roon et al Arthritis and Rheumatism, 2003, 1229-1238). In rheumatoid arthritis they are major contributors to joint inflammation and joint destruction (Conell in N. Eng J. Med. 2004, 350, 2591-2602). Macrophages are also involved in tumour growth and development (Naldini and Carraro in Curr Drug Targets Inflamm Allergy, 2005, 3-8). Hence agents that selectively image macrophage cells could be of value in the diagnosis of cancer, inflammation and autoimmune disease, for example arthritis. Targeting specific cell types would be expected to lead to greater contrast in the imaging results, for example arthritis. The present invention enables a method of targeting imaging agents to macrophages, which is based on the above observation that the way in which the alpha amino acid ester motif is linked to the imaging agent determines whether it is hydrolysed by specific carboxylesterases, and hence whether or not the resultant acid accumulates in different cell types. Specifically, it has been found that the hCE-1 enzyme is primarily expressed in monocytic cells, such as macrophages. In the conjugates of the invention, when the nitrogen of the alpha amino acid ester motif is substituted but not directly bonded to a carbonyl group moiety the ester will only be hydrolysed by hCE-1 and hence the esterase-hydrolysed imaging agent conjugates will only accumulate in cells containing hCE-1 such as monocytic cells, for example macrophages.

The present invention therefore also provides an imaging method for imaging macrophage cells comprising carrying out an imaging study on a subject using the imaging agent of the invention. The method may comprise a step of providing the imaging agent to the subject, or the imaging agent may be pre-delivered.

The present invention also provides the imaging agent of the invention for use as an imaging agent for macrophage cells. The imaging agent may also be for use in a method comprising delivering said agent to a subject and imaging macrophage cells of said subject. Further provided is the use of an imaging agent of the invention in the manufacture of an agent for use in a method comprising delivering said agent to a subject and imaging macrophage cells of said subject.

The invention may be used in a variety of imaging studies including, for example, imaging atherosclerotic plaques or the joints of arthritic patients.

There are of course many possible ester groups which may in principle be present in the carboxylesterase ester motif for attachment to the imaging agent. Likewise, there are many alpha amino acids, both natural and non-natural, differing in the side chains on the alpha carbon, which may be used as esters in the carboxylesterase ester motif. Some alpha amino acid esters are rapidly hydrolysed by one or more of the hCE-1, -2 and -3 isotypes or cells containing these enzymes, while others are more slowly hydrolysed, or hydrolysed only to a very small extent. In general, if the carboxylesterase hydrolyses the free amino acid ester to the parent acid it will, subject to the N-carbonyl dependence of hCE-2 and hCE-3 discussed above, also hydrolyse the amino acid ester motif when covalently conjugated to the imaging agent. Hence, the broken cell assay and/or the isolated carboxylesterase assay described herein provide a straightforward, quick and simple first screen for esters which have the required hydrolysis profile. Amino acid ester motifs selected in that way may then be re-assayed in the same carboxylesterase assay when conjugated to the imaging agent via the chosen conjugation chemistry, to confirm that it is still a carboxylesterase substrate in that background. Suitable types of amino acid esters will be discussed below, but at this point it may be mentioned that it has been found that t-butyl esters of alpha amino acids are relatively poor substrates for hCE-1, -2 and -3, whereas cyclopentyl esters are effectively hydrolysed. Suitable alpha amino acids will also be discussed in more detail below, but at this point it may be mentioned that phenylalanine, homophenylalanine, phenylglycine and leucine are generally suitable, and esters of secondary alcohols are preferred.

As stated above, the alpha amino acid ester may be conjugated to the imaging agent via the amino group of the amino acid ester. A linker radical may be present between the amino acid ester motif and the imaging agent. For example, the alpha amino acid ester may be conjugated to the imaging agent as a radical of formula (IA) or (IB):

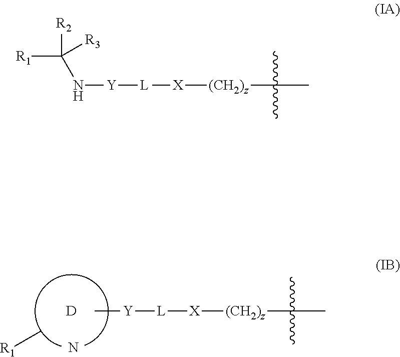

wherein
R₁ is an ester group which is hydrolysable by one or more intracellular carboxylesterase enzymes to a carboxylic acid group;
R₂ is the side chain of a natural or non-natural alpha amino acid;

### R₃ is H or R₂;

Y is a bond, —S(═O)₂—, —C(═S)—NR₄—, —C(═NH)NR₄—, —S(═O)₂NR₄—, or —NR₄—C(═O) wherein R₄ is hydrogen or optionally substituted C₁-C₆ alkyl;
L is a divalent radical of formula -(Alk¹)ₘ(O)ₙ(Alk²)ₚ- wherein
m, n and p are independently 0 or 1,
Q is (i) an optionally substituted divalent mono- or bicyclic carbocyclic or heterocyclic radical having 5-13 ring members, or (ii), in the case where both m and p are 0, a divalent radical of formula —X²-Q¹- or -Q¹-X²— wherein X² is —O—, —S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl;
   X represents a bond, —O—, —C(═O)—, —S(═O)₂—, —NR₄C(═O)—, —C(═O)NR₄—, —NR₄C(═O)NR₅—, —NR₄S(═O)₂—, or —S(═O)₂NR₄— wherein R₄ and R₅ are independently hydrogen or optionally substituted C₁-C₆ alkyl;
   z is 0 or 1; and
   ring D is an optionally substituted 3- to 7-membered heterocyclyl group wherein: R₁ is linked to a ring carbon adjacent to the ring nitrogen shown; and ring D is optionally fused to a second ring comprising a phenyl, 5- to 6-membered heteroaryl, C₃₋₇, carbocyclyl or 5- to 6-membered heterocyclyl group, wherein when the ring D is fused to a second ring comprising a phenyl, 5- to 6-membered heteroaryl, C₃₋₇ carbocyclyl or 5- to 6-membered heterocyclyl group then the linker group —Y-L-X—(CH₂)_{z} may be from a ring atom in ring D or said second ring.

In one aspect of the invention, X represents a bond —C(═O)—, —S(═O)₂—, —NR₄C(═O)—, —C(═O)NR₄—, —NR₄C(═O)NR₅—, —NR₄S(═O)₂—, or —S(═O)₂NR₄— wherein R₄ and R₅ are independently hydrogen or optionally substituted C₁-C₆ alkyl.

In these radicals R₁ is a carboxyl ester group which is hydrolysable by one or more intracellular carboxylesterase enzymes to a carboxylic acid group and the group R₁—C—NH— forms the alpha amino acid group. In the formula (IA) the alpha amino acid is linear and is linked via the amino group to a linker —Y-L-X—(CH₂)_{z}— and thus to the imaging agent. In the formula (IB) the alpha amino acid group is cyclic, with a ring formed between the amino group and the alpha carbon atom, the ring being further linked to the imaging agent via the linker radical —Y-L-X—(CH₂)_{z}—.

The term “ester” or “esterified carboxyl group” means a group R₉O(C═O)— in which R₉ is the group characterising the ester, notionally derived from the alcohol R₉OH.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” means a hydrocarbon chain having from a to b carbon atoms, at least one double bond, and two unsatisfied valences.

As used herein the term “Cₐ-C_{b} alkynyl” wherein a and b are integers refers to straight chain or branched chain hydrocarbon groups having from two to six carbon atoms and having in addition one triple bond. This term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “divalent (Cₐ-C_{b})alkynylene radical” wherein a and b are integers refers to a divalent hydrocarbon chain having from 2 to 6 carbon atoms, and at least one triple bond.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic (e.g. saturated) radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, azepinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, phenyl, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl or monocyclic heteroaryl having 5 or 6 ring atoms. An “optional substituent” may be one of the foregoing substituent groups.

The term “side chain of a natural or non-natural alpha-amino acid” refers to the group R¹ in a natural or non-natural amino acid of formula NH₂—CH(R¹)—COOH.

Examples of side chains of natural or non-natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, α-aminoadipic acid, α-amino-n-butyric acid, 3,4-dihydroxyphenylalanine, homoserine, α-methylserine, ornithine, pipecolic acid, and thyroxine. Preferred side chains include those of L-leucine, L-phenylglycine, L-cyclohexylglycine, L-tButyl serine, dimethyl glycine and alanine

Natural alpha-amino acids which contain functional substituents, for example amino, carboxyl, hydroxy, mercapto, guanidyl, imidazolyl, or indolyl groups in their characteristic side chains include arginine, lysine, glutamic acid, aspartic acid, tryptophan, histidine, serine, threonine, tyrosine, and cysteine. When R₂ in the compounds of the invention is one of those side chains, the functional substituent may optionally be protected.

The term “protected” when used in relation to a functional substituent in a side chain of a natural alpha-amino acid means a derivative of such a substituent which is substantially non-functional. For example, carboxyl groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or NHC(═O)OCH₂Ph carbamate), hydroxyl groups may be converted to ethers (for example an OC₁-C₆alkyl or a O(C₁-C₈ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

Examples of side chains of non-natural alpha amino acids include those referred to below in the discussion of suitable R₂ groups for use in compounds of the present invention.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulfonic, glutamic, lactic, and mandelic acids and the like.

### The Ester Group R₁

In addition to the requirement that the ester group must be hydrolysable by one or more intracellular enzymes, it may be preferable for some applications (for example for systemic administration of the conjugate) that it be resistant to hydrolysis by carboxylester-hydrolysing enzymes in the plasma, since this ensures the conjugated modulator will survive after systemic administration for long enough to penetrate cells as the ester. It is a simple matter to test any given conjugate to measure its plasma half life as the ester, by incubation in plasma. However, it has been found that esters notionally derived from secondary alcohols are more stable to plasma carboxylester-hydrolysing enzymes than those derived from primary alcohols. Furthermore, it has also been found that although esters notionally derived from tertiary alcohols are generally stable to plasma carboxylester-hydrolysing enzymes, they are often also relatively stable to intracellular carboxylesterases. Taking these findings into account, it is presently preferred that R₁ in formulae (IA) and (IB) above, is an ester group of formula —(C═O)OR₁₄ wherein R₁₄ is
R₈R₉R₁₀C— wherein
(i) R₈ is hydrogen or optionally substituted (C₁-C₃)alkyl-(Z¹)ₐ—[(C₁-C₃)alkyl]_{b}— or (C₂-C₃)alkenyl-(Z¹)ₐ-[(C₁-C₃)alkyl]_{b}— wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic or bridged monocyclic carbocyclic ring of from 3 to 7 ring atoms or bicyclic or bridged monocyclic carbocyclic ring system of 8 to 10 ring atoms, and R₁₀ is hydrogen.

In cases (i), (ii) and (iii) above, “alkyl” includes fluoroalkyl.

Within these classes (i), (ii) and (iii), R₁₀ is often hydrogen. Specific examples of R₁₄ include methyl, trifluoromethyl, ethyl, n- or iso-propyl, n-, sec- or tert-butyl, neopentyl, cyclohexyl, cyclopentyl, norbornyl, allyl, phenyl, benzyl, 2-, 3- or 4-pyridylmethyl, N-methylpiperidin-4-yl, tetrahydrofuran-3-yl or methoxyethyl, for example methyl, trifluoromethyl, ethyl, n- or iso-propyl, n-, sec- or tert-butyl, cyclohexyl, norbornyl, allyl, phenyl, benzyl, 2-, 3- or 4-pyridylmethyl, N-methylpiperidin-4-yl, tetrahydrofuran-3-yl or methoxyethyl. Currently preferred is where R₁₄ is neopentyl or cyclopentyl, in particular cyclopentyl.

### The Amino Acid Side Chain R₂

Subject to the requirement that the ester group R₁ be hydrolysable by intracellular carboxylesterase enzymes, the selection of the side chain group R₂ can determine the rate of hydrolysis. For example, when the carbon in R₂ adjacent to the alpha amino acid carbon does not contain a branch eg when R₂ is ethyl, isobutyl or benzyl the ester is more readily hydrolysed than when R₂ is branched eg isopropyl or t-butyl.

Examples of amino acid side chains include

C₁-C₆ alkyl, phenyl, 2,- 3-, or 4-hydroxyphenyl, 2,- 3-, or 4-methoxyphenyl, 2,- 3-, or 4-pyridylmethyl, benzyl, phenylethyl, 2-, 3-, or 4-hydroxybenzyl, 2,- 3-, or 4-benzyloxybenzyl, 2,- 3-, or 4-C₁-C₆ alkoxybenzyl, and benzyloxy(C₁-C₆alkyl)- groups;
the characterising group of a natural a amino acid, in which any functional group may be protected;
groups -[Alk]ₙR₆ where Alk is a (C₁-C₆)alkyl or (C₂-C₆)alkenyl group optionally interrupted by one or more —O—, or —S— atoms or —N(R₇)— groups [where R₇ is a hydrogen atom or a (C₁-C₆)alkyl group], n is 0 or 1, and R₆ is an optionally substituted cycloalkyl or cycloalkenyl group;
a benzyl group substituted in the phenyl ring by a group of formula —OCH₂COR₈ where R₈ is hydroxyl, amino, (C₁-C₆)alkoxy, phenyl(C₁-C₈)alkoxy, (C₁-C₆)alkylamino, di((C₁-C₈)alkyl)amino, phenyl(C₁-C₆)alkylamino, the residue of an amino acid or acid halide, ester or amide derivative thereof, said residue being linked via an amide bond, said amino acid being selected from glycine, α or β alanine, valine, leucine, isoleucine, phenylalanine, tyrosine, tryptophan, serine, threonine, cysteine, methionine, asparagine, glutamine, lysine, histidine, arginine, glutamic acid, and aspartic acid;
a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₈)alkylamino, mercapto, (C₁-C₆)alkylthio, hydroxy(C₁-C₆)alkyl, mercapto(C₁-C₆)alkyl or (C₁-C₆)alkylphenylmethyl; and
a group —CRₐR^{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R^{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆) alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

Examples of particular R₂ groups include benzyl, phenyl, cyclohexylmethyl, pyridin-3-ylmethyl, tert-butoxymethyl, iso-butyl, sec-butyl, tert-butyl, 1-benzylthio-1-methylethyl, 1-methylthio-1-methylethyl, and 1-mercapto-1-methylethyl, phenylethyl. Presently preferred R₂ groups include phenyl, benzyl, tert-butoxymethyl, phenylethyl and iso-butyl.

The Radical —Y-L-X—[CH₂]_{z}—

When the alpha amino acid ester is conjugated to the imaging agent by a radical of formula (IA) or (IB) this radical (or bond) arises from the particular chemistry strategy chosen to link the amino acid ester motif R₁C(R₃)(R₂)NH—, or the cyclic amino acid group in the case of formula (IB), to the imaging agent. Clearly the chemistry strategy for that coupling may vary widely, and thus many combinations of the variables Y, L, X and z are possible.

It should also be noted that the benefits of the alpha amino acid ester motif described above (facile entry into the cell, carboxylesterase hydrolysis within the cell, and accumulation within the cell of the carboxylic acid hydrolysis product) are best achieved when the linkage between the amino acid ester motif and the imaging agent is not a substrate for peptidase activity within the cell, which might result in cleavage of the amino acid from the molecule. Of course, stability to intracellular peptidases is easily tested by incubating the compound with disrupted cell contents, and analysing for any such cleavage.

With the foregoing general observations in mind, taking the variables making up the radical —Y-L-X—[CH₂]_{z} in turn:
z may be 0 or 1, so that a methylene radical linked to the modulator is optional.
when the alpha amino acid ester is conjugated to the inhibitor as a radical of formula (IA) or (IB), Y is for example selected from a bond, —S(═O)₂—, —C(═S)—NR₄—, —C(═NH)—NR₄— and —S(═O)₂NR₄, wherein R₄ is hydrogen or optionally substituted C₁-C₆ alkyl; and preferably Y is a bond.
In the radical L, examples of Alk¹ and Alk² radicals, when present, include —CH₂—, —CH₂CH₂— —CH₂CH₂CH₂—, —CH₂CH₂CH₂CH₂—, —CH═CH—, —CH═CHCH₂—, —CH₂CH═CH—, CH₂CH═CHCH₂—, —C≡C—, —C≡CCH₂—, CH₂C≡C—, and CH₂C≡CCH₂. Additional examples of Alk¹ and Alk² include —CH₂W—, —CH₂CH₂W—, —WCH₂CH₂W—, —CH₂CH₂WCH₂—, —CH₂CH₂WCH(CH₃)—, —CH₂WCH₂CH₂—, —CH₂WCH₂CH₂WCH₂—, and —WCH₂CH₂— where W is —O—, —S—, —NH—, —N(CH₃)—, or —CH₂CH₂N(CH₂CH₂OH)CH₂—; for example —CH₂W—, —CH₂CH₂W—, —CH₂CH₂WCH₂—, —CH₂CH₂WCH(CH₃)—, —CH₂WCH₂CH₂—, —CH₂WCH₂CH₂WCH₂—, and —WCH₂CH₂— where W is —O—, —S—, —NH—, —N(CH₃)—, or —CH₂CH₂N(CH₂CH₂OH)CH₂—. Further examples of Alk¹ and Alk² include divalent cyclopropyl, cyclopentyl and cyclohexyl radicals.
In L, when n is 0, the radical is a hydrocarbon chain (optionally substituted and perhaps having an ether, thioether or amino linkage). Presently it is preferred that there be no optional substituents in L. When both m and p are 0, L is a divalent mono- or bicyclic carbocyclic or heterocyclic radical with 5-13 ring atoms (optionally substituted). When n is 1 and at least one of m and p is 1, L is a divalent radical including a hydrocarbon chain or chains and a mono- or bicyclic carbocyclic or heterocyclic radical with 5-13 ring atoms (optionally substituted). When present, Q may be, for example, a divalent phenyl, naphthyl, cyclopropyl, cyclopentyl, or cyclohexyl radical, or a mono-, or bi-cyclic heterocyclic radical having 5 to 13 ring members, such as piperidinyl, piperazinyl, indolyl, pyridyl, thienyl, or pyrrolyl radical, but 1,4-phenylene is presently preferred.
Specifically, in some embodiments of the invention, m and p may be 0 with n being 1. In other embodiments, n and p may be 0 with m being 1. In further embodiments, m, n and p may be all 0. In still further embodiments m may be 0, n may be 1 with Q being a monocyclic heterocyclic radical, and p may be 0 or 1. In still further embodiments, m and n may both equal 1 with Q being a monocyclic heterocyclic radical and p being 0 or 1. Alk¹ and Alk², when present, may be selected from —CH₂—, —CH₂CH₂—, and —CH₂CH₂CH₂— and Q may be 1,4-phenylene. Alternatively, Alk¹ and Alk², when present, may be selected from —(CH₂)—, -Het¹-(CH₂)ₓ-(Het¹)_{y}- and —CH═CH—, wherein x is 0, 1, 2 or 3, y is 0 or 1 and Het¹ is —O— or —NH—, in particular from —CH₂—, —CH₂CH₂—, —CH₂CH₂CH₂—, —O—CH₂—, —O(CH₂)₂—, —O(CH₂)₂O—, —O(CH₂)₂NH— and —CH═CH— and Q may be 1,4-phenylene. In particular, Alk¹ may be methylene.

X may represent a bond, —O— or —NR₄C(═O)—, wherein the N is linked to the group Y and wherein R₄ represents hydrogen or methyl, preferably hydrogen.

Specific examples of the radical —Y-L-X—[CH₂]_{z}— include —(CH₂)ᵥ—, —(CH₂)ᵥO—, (CH₂)_{w}O—

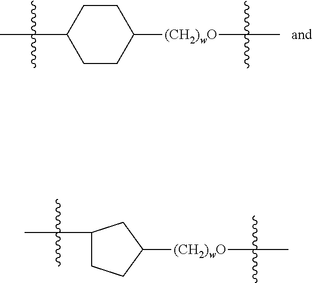

wherein v is 1, 2, 3 or 4 and w is 1, 2 or 3, such as —CH₂—, —CH₂O—.

Further examples of the radical —Y-L-X—[CH₂]_{z}— include —(CH₂)ₓ-Het²-, —CH₂-Ph-(Het¹), —(CH₂)ₓ-(Het²)_{y}- and —CH₂-Ph-CH═CH—, wherein Ph is a 1,4-phenylene group, Het¹ is —O— or —NH—, Het² is —O—, —NH— or —NHC(═O)—, x is 0, 1 or 2, y is 0 or 1 and yy is 0 or 1. In one embodiment, x, y and yy are all 0. Alternatively, y may be 1 and x may be 0, 1 or 2, whilst yy is 0 or 1. In a further embodiment, y is 0, x is 1 or 2 and yy is 1. Specific examples of the radical —Y-L-X—[CH₂]_{z}— include —CH₂-Ph-O—(CH)₂—, —CH₂-Ph-O—(CH)₂—O—, —CH₂-Ph-O—(CH)₂—NH—, —CH₂—, —CH₂CH₂—, —CH₂-Ph-, —CH₂-Ph-O—, —CH₂-Ph-CH═CH—, —CH₂-Ph-(CH)₂—N—C(═O)— and —(CH)₂—N—C(═O)—.

As discussed above, the ester part of the alpha amino acid ester conjugates of the invention is selectively hydrolysed to the corresponding carboxylic acid part by the intracellular carboxylesterase hCE-1 relative to hCE-2 and hCE-3. For example, the ester part may be hydrolysed at least 2 times faster in the broken cell assay (discussed below) based on monocytic cells than in the same assay using non-monocytic cells. Preferably the imaging agent conjugates are hydrolysed at least 3 times faster in monocytic cells relative to non-monocytic cells; more preferably at least 5 times faster; still more preferably at least 10 times faster; and yet more preferably at least 20 times faster.

For compounds of the invention which are to be administered systemically, esters with a slow rate of carboxylesterase cleavage are preferred, since they are less susceptible to pre-systemic metabolism. Their ability to reach their target tissue intact is therefore increased, and the ester can be converted inside the cells of the target tissue into the acid product. However, for local administration, where the ester is either directly applied to the target tissue or directed there by, for example, inhalation, it will often be desirable that the ester has a rapid rate of esterase cleavage, to minimise systemic exposure and consequent unwanted side effects. Where the esterase motif is linked to the imaging agent via its amino group, as in formula (IA) or (IB) above, if the carbon adjacent to the alpha carbon of the alpha amino acid ester is monosubstituted, ie R₂ is CH₂R^{z} (R^{z} being the mono-substituent) then the esters tend to be cleaved more rapidly than if that carbon is di- or tri-substituted, as in the case where R₂ is, for example, phenyl or cyclohexyl.

ring D

In the case of formula (IB), the ring D is typically a non-fused 3- to 7-membered heteroaryl or heterocyclyl group where R₁ is linked to a carbon atom adjacent the nitrogen atom shown in ring D. Typically, ring D is a non-fused 5- to 7-membered heteroaryl or heterocyclyl group, preferably a non-fused 5- or 6-membered heteroaryl or heterocyclyl group. More preferably ring D is a non-fused 5- to 6-membered heterocyclyl group, for example a saturated, non-fused 5- to 6-membered heterocyclyl group containing, in addition to the nitrogen atom depicted in ring D, none, one or two heteroatoms selected from N, O and S. Examples of suitable groups for ring D include pyrrolidinyl, oxazolidinyl, isoxazolidinyl, imidazolidinyl, pyrazolidinyl, thiazolidinyl, isothiazolidinyl, piperidinyl, hexahydropyrimidinyl, piperazinyl, morpholinyl and thiomorpholinyl groups. More preferably Ring D is a pyrrolidinyl, piperazinyl or piperidinyl group, more preferably a piperidyl or piperazinyl group, in particular a piperazinyl group.

The ring D may be connected to group Y via a carbon atom in the ring D, via a ring fused to the ring D or, in the case that an additional nitrogen atom is present, via said additional nitrogen atom. Preferably the ring D is connected to the group Y via a carbon atom in the ring D or via an additional nitrogen atom. In one embodiment, ring D is a piperazinyl ring wherein one nitrogen atom forms a part of the amino acid group and the other nitrogen atom is connected to the group Y.

In addition to bearing group R₁, ring D may bear a group R₂ on the same carbon atom which carries the group R₁. Suitable groups R₂ are those described above. In one embodiment, R₂ is not present and the ring carbon atom carrying R₁ is not further substituted. In addition to the R₁ and R₂ groups, ring D is preferably unsubstituted or substituted by 1 or 2 groups selected from halogen atoms and C₁₋₄ alkyl, C₁₋₄ alkoxy and hydroxyl groups. More preferably ring D, apart from bearing the groups R₁ and optionally R₂, is unsubstituted.

Preferred ring D groups are the radicals (a), (b) and (c), most preferably (c):

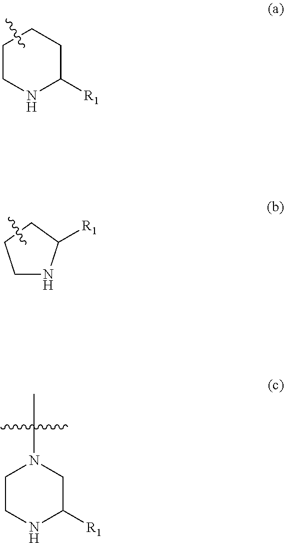

In one embodiment of the invention, an imaging agent is conjugated to the alpha mono- or di-substituted amino acid ester by conjugation to a radical as shown in formula (IA):

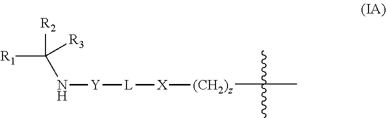

wherein
R₁ is an ester group which is hydrolysable by one or more intracellular carboxylesterase enzymes to a carboxylic acid group;
R₂ is the side chain of a natural or non-natural alpha amino acid;

### R₃ is H or R₂;

Y is a bond, —S(═O)₂—, —C(═S)—NR₄—, —C(═NH)NR₄—, —S(═O)₂NR₄—, or —NR₄—C(═O) wherein R₄ is hydrogen or optionally substituted C₁-C₈ alkyl;
L is a divalent radical of formula -(Alk¹)ₘ(Q)ₙ(Alk²)ₚ- wherein
m, n and p are independently 0 or 1,
Q is (i) an optionally substituted divalent mono- or bicyclic carbocyclic or heterocyclic radical having 5-13 ring members, or (ii), in the case where both m and p are 0, a divalent radical of formula —X²-Q¹- or -Q¹-X²— wherein X² is —O—, —S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl;
   X represents a bond, —O—, —C(═O)—, —S(═O)₂—, —NR₄C(═O)—, —C(═O)NR₄—, —NR₄C(═O)NR₅—, NR₄S(═O)₂—, or —S(═O)₂NR₄— wherein R₄ and R₅ are independently hydrogen or optionally substituted C₁-C₆ alkyl;
   z is 0 or 1.

In the foregoing embodiment R₃ is often H.

In another embodiment, R₁ is ester group of formula —(C═O)OR₁₄ wherein R₁₄ is
R₈R₉R₁₀C— wherein
(i) R₈ is hydrogen or optionally substituted (C₁-C₃)alkyl-(Z¹)ₐ—[(C₁-C₃)alkyl]_{b}— or (C₂-C₃)alkenyl-(Z¹)ₐ—[(C₁-C₃)alkyl]_{b}— wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic or bridged monocyclic carbocyclic ring of from 3 to 7 ring atoms or bicyclic or bridged monocyclic carbocyclic ring system of 8 to 10 ring atoms, and R₁₀ is hydrogen;
wherein in cases (i), (ii) and (iii) above, “alkyl” includes fluoroalkyl;
   R₂ is selected from:
   C₁-C₆ alkyl, phenyl, 2,- 3-, or 4-hydroxyphenyl, 2,- 3-, or 4-methoxyphenyl, 2,- 3-, or 4-pyridylmethyl, benzyl, phenylethyl, 2-, 3-, or 4-hydroxybenzyl, 2,- 3-, or 4-benzyloxybenzyl, 2,- 3-, or 4-C₁-C₆ alkoxybenzyl, and benzyloxy(C₁-C₆ alkyl)- groups;
   the characterising group of a natural ca amino acid, in which any functional group may be protected;
   groups -[Alk]ₙR₆ where Alk is a (C₁-C₆)alkyl or (C₂-C₆)alkenyl group optionally interrupted by one or more —O—, or —S— atoms or —N(R₇)— groups [where R₇ is a hydrogen atom or a (C₁-C₆)alkyl group], n is 0 or 1, and R₆ is an optionally substituted cycloalkyl or cycloalkenyl group;
   a benzyl group substituted in the phenyl ring by a group of formula —OCH₂COR₈ where R₈ is hydroxyl, amino, (C₁-C₆)alkoxy, phenyl(C₁-C₆)alkoxy, (C₁-C₆)alkylamino, di((C₁-C₆)alkyl)amino, phenyl(C₁-C₆)alkylamino, the residue of an amino acid or acid halide, ester or amide derivative thereof, said residue being linked via an amide bond, said amino acid being selected from glycine, α or β alanine, valine, leucine, isoleucine, phenylalanine, tyrosine, tryptophan, serine, threonine, cysteine, methionine, asparagine, glutamine, lysine, histidine, arginine, glutamic acid, and aspartic acid;
   a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; and
   a group —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆)alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl;

### R₃ is H or R₂;

Y is a bond;
L is a divalent radical of formula -(Alk¹)ₘ(Q)ₙ(Alk²)ₚ— wherein
m, n and p are independently 0 or 1,
Q is 1,4-phenylene;
Alk¹ and Alk² independently represent —CH₂—, —CH₂CH₂— —CH₂CH₂CH₂—, —CH₂CH₂CH₂CH₂—, —CH═CH—, —CH═CHCH₂—, —CH₂CH═CH—, CH₂CH═CHCH₂—, —C≡C—, —C≡CCH₂—, CH₂C≡C—, CH₂C≡CCH₂, —CH₂W—, —CH₂CH₂W—, —WCH₂CH₂W—, —CH₂CH₂WCH₂—, —CH₂CH₂WCH(CH₃)—, —CH₂WCH₂CH₂—, —CH₂WCH₂CH₂WCH₂—, or —WCH₂CH₂— where W is —O—, —S—, —NH—, —N(CH₃)—, or —CH₂CH₂N(CH₂CH₂OH)CH₂—, or Alk¹ and Alk² may independently represent cyclopropyl, cyclopentyl or cyclohexyl radicals;
   X represents a bond, —O—, or —NHC(═O)—; and
   z is 0 or 1.

In another embodiment, R₁ is an ester group COOR₁₄, wherein R₁₄ is selected from methyl, trifluoromethyl, ethyl, n- or iso-propyl, n-, sec- or tert-butyl, neopentyl, cyclohexyl, cyclopentyl, norbornyl, allyl, phenyl, benzyl, 2-, 3- or 4-pyridylmethyl, N-methylpiperidin-4-yl, tetrahydrofuran-3-yl or methoxyethyl;

R₂ is selected from benzyl, phenyl, cyclohexylmethyl, pyridin-3-ylmethyl, tert-butoxymethyl, iso-butyl, sec-butyl, tert-butyl, 1-benzylthio-1-methylethyl, 1-methylthio-1-methylethyl, and 1-mercapto-1-methylethyl, phenylethyl;

### R₃ is H or R₂;

the radical —Y-L-X—(CH₂)_{z} is selected from —(CH₂)ᵥ—, —(CH₂)ᵥO—, (CH₂)_{w}O—

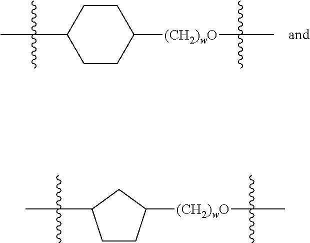

wherein v is 1, 2, 3 or 4 and w is 1, 2 or 3, and from —(CH₂)ₓ-Het²-, —CH₂-Ph-(Het¹), —(CH₂)ₓ-(Het²)_{y}- and —CH₂-Ph-CH═CH—, wherein Ph is a 1,4-phenylene group, Het¹ is —O— or —NH—, Het² is —O—, —NH— or —NHC(═O)—, x is 0, 1 or 2, y is 0 or 1 and yy is 0 or 1.

In the embodiments above, the imaging agent of the invention is therefore of formula (IA′):

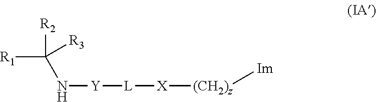

wherein R₁, R₂, R₃, Y, L, X and z are as defined above and Im is an imaging agent.

In another embodiment, an imaging agent is conjugated to the alpha mono- or di-substituted amino acid ester by conjugation to a radical as shown in formula (IB):

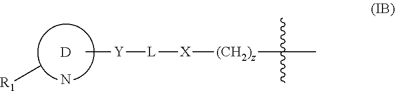

wherein:
ring D is a saturated, non-fused 5- to 6-membered heterocyclyl group containing, in addition to the nitrogen atom depicted in ring D, none, one or two heteroatoms selected from N, O and S, the ring being substituted with a group R¹ and optionally a group R², wherein R₁ is linked to a ring carbon atom adjacent the ring nitrogen atom shown, and wherein R² is carried on the same carbon atom which carries the group R¹, and wherein the ring D is further unsubstituted or substituted by 1 or 2 groups selected from halogen atoms and C₁₋₄ alkyl, C₁₋₄ alkoxy and hydroxyl groups;
R₁ is ester group of formula —(C═O)OR₁₄ wherein R₁₄ is
R₈R₉R₁₀C— wherein
(i) R₈ is hydrogen or optionally substituted (C₁-C₃)alkyl-(Z¹)ₐ—[(C₁-C₃)alkyl]_{b}— or (C₂-C₃)alkenyl-(Z¹)ₐ—[(C₁-C₃)alkyl]_{b}— wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic or bridged monocyclic carbocyclic ring of from 3 to 7 ring atoms or bicyclic or bridged monocyclic carbocyclic ring system of 8 to 10 ring atoms, and R₁₀ is hydrogen;
wherein in cases (i), (ii) and (iii) above, “alkyl” includes fluoroalkyl;
   R₂, when present, is selected from:
   C₁-C₆ alkyl, phenyl, 2,- 3-, or 4-hydroxyphenyl, 2,- 3-, or 4-methoxyphenyl, 2,- 3-, or 4-pyridylmethyl, benzyl, phenylethyl, 2-, 3-, or 4-hydroxybenzyl, 2,- 3-, or 4-benzyloxybenzyl, 2,- 3-, or 4-C₁-C₆ alkoxybenzyl, and benzyloxy(C₁-C₆ alkyl)- groups;
   the characterising group of a natural a amino acid, in which any functional group may be protected;
   groups -[Alk]ₙR₆ where Alk is a (C₁-C₆)alkyl or (C₂-C₆)alkenyl group optionally interrupted by one or more —O—, or —S— atoms or —N(R₇)— groups [where R₇ is a hydrogen atom or a (C₁-C₆)alkyl group], n is 0 or 1, and R₆ is an optionally substituted cycloalkyl or cycloalkenyl group;
   a benzyl group substituted in the phenyl ring by a group of formula —OCH₂COR₈ where R₈ is hydroxyl, amino, (C₁-C₆)alkoxy, phenyl(C₁-C₆)alkoxy, (C₁-C₆)alkylamino, di((C₁-C₆)alkyl)amino, phenyl(C₁-C₆)alkylamino, the residue of an amino acid or acid halide, ester or amide derivative thereof, said residue being linked via an amide bond, said amino acid being selected from glycine, α or β alanine, valine, leucine, isoleucine, phenylalanine, tyrosine, tryptophan, serine, threonine, cysteine, methionine, asparagine, glutamine, lysine, histidine, arginine, glutamic acid, and aspartic acid;
   a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; and
   a group —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆)alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl;
   Y is a bond;
   L is a divalent radical of formula -(Alk¹)ₘ(Q)ₙ(Alk²)ₚ- wherein
m, n and p are independently 0 or 1,
Q is 1,4-phenylene;
Alk¹ and Alk² independently represent —CH₂—, —CH₂CH₂— —CH₂CH₂CH₂—, —CH₂CH₂CH₂CH₂—, —CH═CH—, —CH═CHCH₂—, —CH₂CH═CH—, CH₂CH═CHCH₂—, —C≡C—, —C≡CCH₂—, CH₂C≡C—, CH₂C≡CCH₂, —CH₂W—, —CH₂CH₂W—, —WCH₂CH₂W—, —CH₂CH₂WCH₂—, —CH₂CH₂WCH(CH₃)—, —CH₂WCH₂CH₂—, —CH₂WCH₂CH₂WCH₂—, or —WCH₂CH₂— where W is —O—, —S—, —NH—, —N(CH₃)—, or —CH₂CH₂N(CH₂CH₂OH)CH₂—, or Alk¹ and Alk² may independently represent cyclopropyl, cyclopentyl or cyclohexyl radicals;
   X represents a bond, —O—, or —NHC(═O)—; and
   z is 0 or 1.

Typically, in the above embodiment, R₂ is absent.

In another embodiment,

ring D is a group selected from

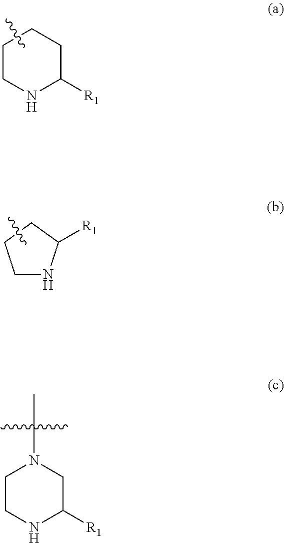

R₁ is an ester group COOR₁₄, wherein R₁₄ is selected from methyl, trifluoromethyl, ethyl, n- or iso-propyl, n-, sec- or tert-butyl, neopentyl, cyclohexyl, cyclopentyl, norbornyl, allyl, phenyl, benzyl, 2-, 3- or 4-pyridylmethyl, N-methylpiperidin-4-yl, tetrahydrofuran-3-yl or methoxyethyl;
the radical —Y-L-X—(CH₂)_{z} is selected from —(CH₂)ᵥ—, —(CH₂)ᵥO—, (CH₂)_{w}O—

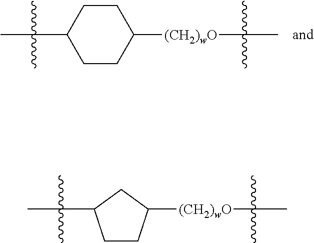

wherein v is 1, 2, 3 or 4 and w is 1, 2 or 3, and from —(CH₂)ₓ-Het²-, —CH₂-Ph-(Het)_{y}, —(CH₂)ₓ-(Het²)_{y}- and —CH₂-Ph-CH═CH—, wherein Ph is a 1,4-phenylene group, Het¹ is —O— or —NH—, Het² is —O—, —NH— or —NHC(═O)—, x is 0, 1 or 2, y is 0 or 1 and yy is 0 or 1.

In the radicals of formula (IB), the radical —Y-L-X—(CH₂)_{z} is most preferably an alkylene group —(CH₂)ᵥ— wherein v is 0, 1, 2, 3 or 4 or a group —(CH₂)-Ph- wherein Ph is a 1,4-phenylene group, more preferably an alkylene group —(CH₂)ᵥ—.

In the embodiments above where the amino acid radical is conjugated as a radical of formula (IB), the imaging agent of the invention is of formula (IB′):

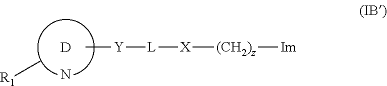

wherein R₁, D, Y, L, X and z are as defined above and Im is an imaging agent.

Specific examples of imaging agents according to the invention are the compounds of the formula:

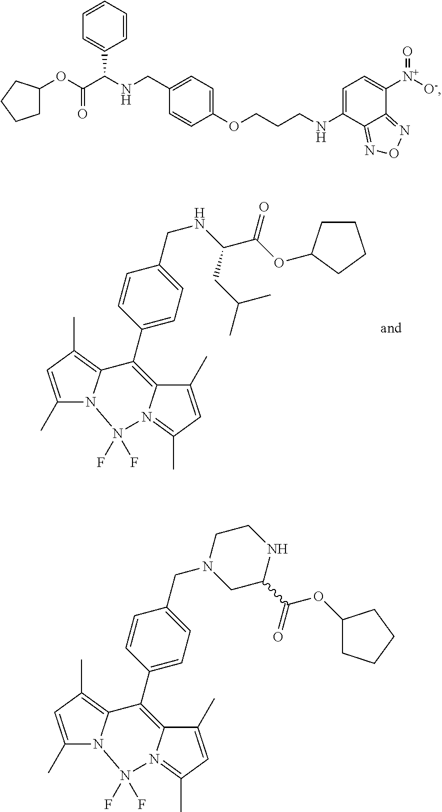

and salts thereof.

Preferably the imaging agents described herein are for use as imaging agents for macrophage cells.

### Imaging Agents

The principles of this invention can be applied to a wide range of imaging agents. An appropriate alpha amino acid ester motif may be linked directly or indirectly to any one of a wide range of imaging agents. For example, Table 1 lists some suitable imaging agents.

TABLE 1

| Imaging Agent | Number | Utility | Source | Structure |
|---|---|---|---|---|
| Gadopentetate dimeglumine | 1 | MRI contrast agent | Schering AG | 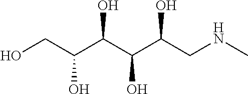 |
| | | | | 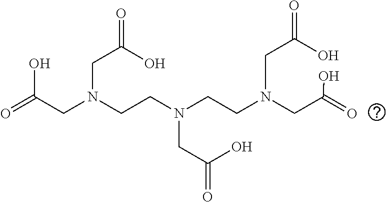 [SMILES: O=C(O)CN(CCN(CC(=O)O)CC(=O)O)CCN(CC(=O)O)CC(=O)O] |
| Gadoversetamide | 2 | MRI contrast agent | Mallinckrodt Inc | 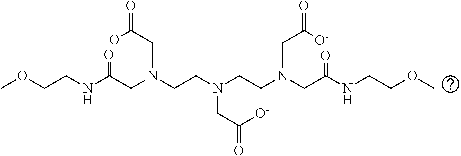 |
| Iopamidol | 3 | X-ray contrast agent | Bracco SpA | 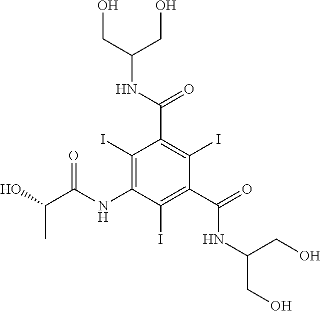 [SMILES: C[C@H](O)C(=O)Nc1c(I)c(C(=O)NC(CO)CO)c(I)c(C(=O)NC(CO)CO)c1I] |
| Iohexol | 4 | X-ray contrast agent | GE Healthcare | 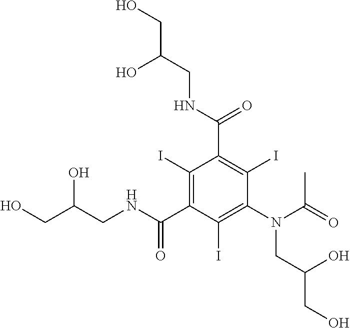 [SMILES: CC(=O)N(CC(O)CO)c1c(I)c(C(=O)NCC(O)CO)c(I)c(C(=O)NCC(O)CO)c1I] |
| Iobitridol | 5 | Xray contrast agent | Guerbet | 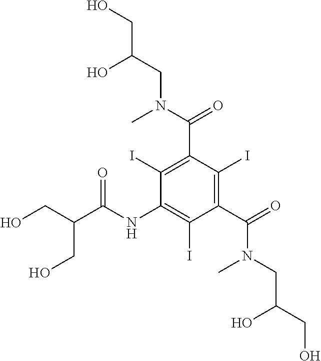 [SMILES: CN(CC(O)CO)C(=O)c1c(I)c(NC(=O)C(CO)CO)c(I)c(C(=O)N(C)CC(O)CO)c1I] |
| Iomeprol | 6 | X-ray contrast agent | Bracco SpA | 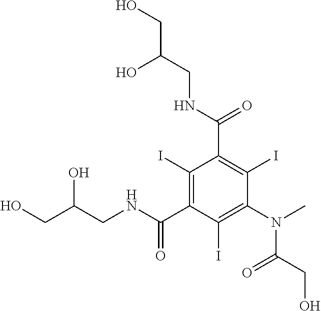 [SMILES: CN(C(=O)CO)c1c(I)c(C(=O)NCC(O)CO)c(I)c(C(=O)NCC(O)CO)c1I] |
| Oxilan | 7 | X-ray contrast agent | Cook Imaging Inc | 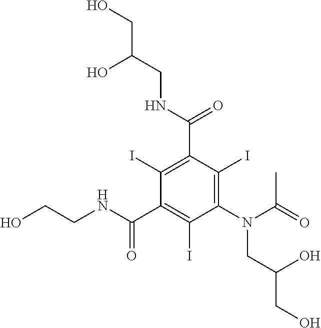 [SMILES: CC(=O)N(CC(O)CO)c1c(I)c(C(=O)NCCO)c(I)c(C(=O)NCC(O)CO)c1I] |
| Iopanoic acid | 8 | X-ray contrast agent | Various | 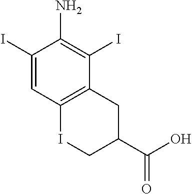 |
| Iodamide | 9 | X-ray contrast agent | Various | 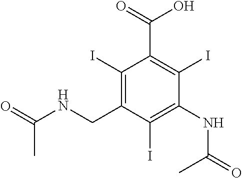 [SMILES: CC(=O)NCc1c(I)c(NC(C)=O)c(I)c(C(=O)O)c1I] |
| Iopromide | 10 | X-ray contrast agent | Schering AG | 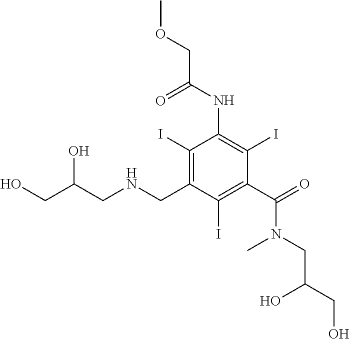 [SMILES: COCC(=O)Nc1c(I)c(CNCC(O)CO)c(I)c(C(=O)N(C)CC(O)CO)c1I] |
| Technetium Tc 99m bisicate | 11 | SPECT imaging agent | E I DuPont de Nemours & Co | 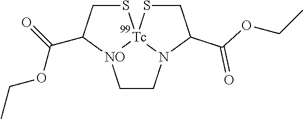 |
| 123I-Ioflupane | 12 | SPECT | GE Healthcare | 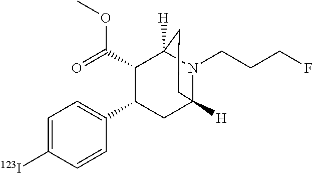 [SMILES: COC(=O)[C@H]1[C@@H](c2ccc([123I])cc2)C[C@@H]2CC[C@H]1N2CCCF] |
| 123I-Iofetamine | 13 | SPECT agent | Roche Holding AG | 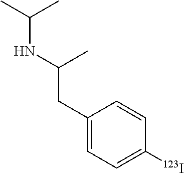 |
| Flurpiridaz-F18 | 14 | PET imaging agent | Lantheus Medical Imaging Inc | 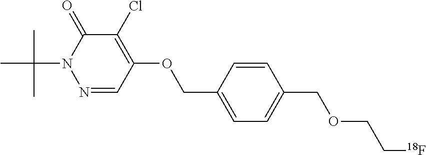 [SMILES: CC(C)(C)n1ncc(OCc2ccc(COCC[18F])cc2)c(Cl)c1=O] |
| Florbetapir-F18 | 15 | PET imaging agent | University of Michigan | 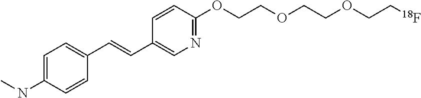 [SMILES: CNc1ccc(/C=C/c2ccc(OCCOCCOCC[18F])nc2)cc1] |
| Presently unnamed | 16 | Fluorescence Imaging agent | See: Chongzhao Ran et al. J. Am. Chem. Soc., 2009, 131 (42), pp 15257-15261 | 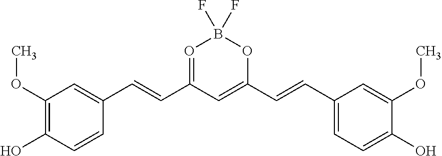 |
| Presently unnamed | 17 | Fluorescence Imaging agent | See: US4774339 | 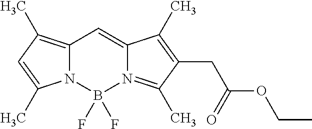 |
| Presently unnamed | 18 | Fluorescence Imaging agent | See: Merkel L et al., Chembiochem, 2010, 11(3), 305-14. | 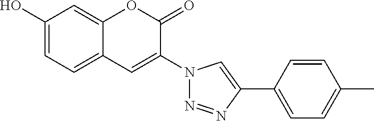 [SMILES: Cc1ccc(-c2cn(-c3cc4ccc(O)cc4oc3=O)nn2)cc1] |

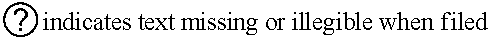

The imaging agents listed in Table 1 are merely exemplary of the imaging agents available for use in the present invention. Variations of the above imaging agents or alternative imaging agents are also envisaged. In particular, some commercially available imaging agents may incorporate additional functional groups. Such functional groups may be absent in the imaging agent of the invention.

For the purpose of illustration, reference is now made to Table 2, which lists examples illustrating how an alpha amino acid ester may be linked, directly or indirectly to a known imaging agent.

TABLE 2

| | Imaging | | |
|---|---|---|---|
| | agent as | | |
| Reference | rererenced | | |
| Number | in Table 1 | Utility | Structure |
| 1 | 14 | PET | 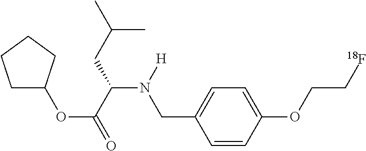 [SMILES: CC(C)C[C@H](NCc1ccc(OCC[18F])cc1)C(=O)OC1CCCC1] |
| 2* | — | PET | 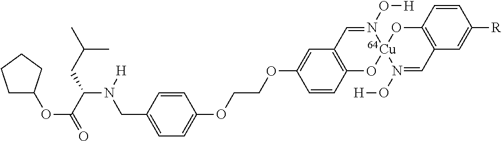 |
| 3 | 13 | SPECT | 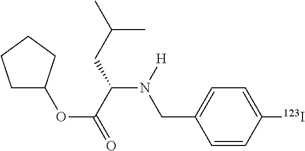 [SMILES: CC(C)C[C@H](NCc1ccc([123I])cc1)C(=O)OC1CCCC1] |
| 4 | 13 | SPECT | 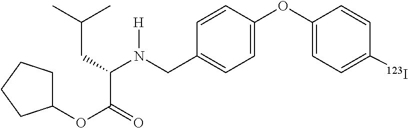 [SMILES: CC(C)C[C@H](NCc1ccc(Oc2ccc([123I])cc2)cc1)C(=O)OC1CCCC1] |
| 5 | 17 | Fluorescence | 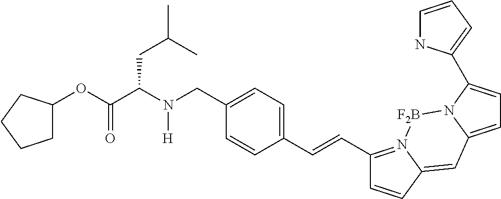 |
| 6 | 17 | Fluorescence | 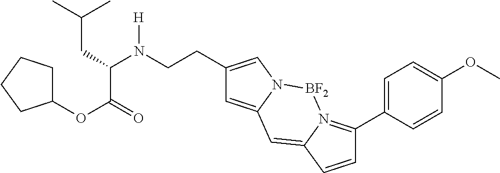 |
| 7 | 17 | Fluorescence | 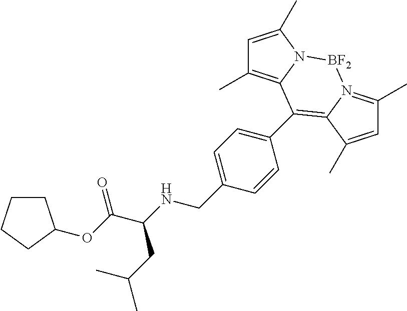 |
| 8 | 16 | Fluorescence | 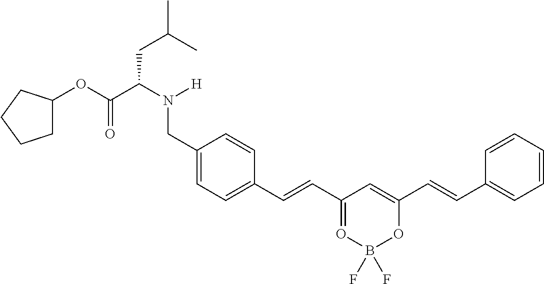 |
| 9 | 18 | Fluorescence | 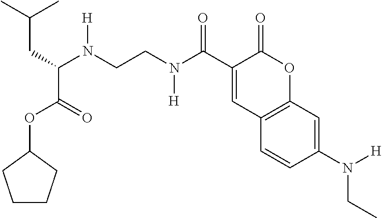 [SMILES: CCNc1ccc2cc(C(=O)NCCN[C@@H](CC(C)C)C(=O)OC3CCCC3)c(=O)oc2c1] |
| 10 | 18 | Fluorescence | 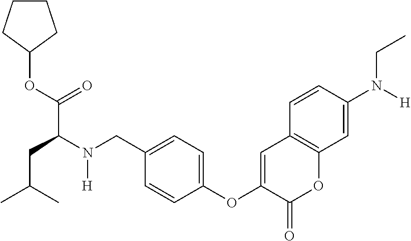 [SMILES: CCNc1ccc2cc(Oc3ccc(CN[C@@H](CC(C)C)C(=O)OC4CCCC4)cc3)c(=O)oc2c1] |
| 11 | 2 | MRI | 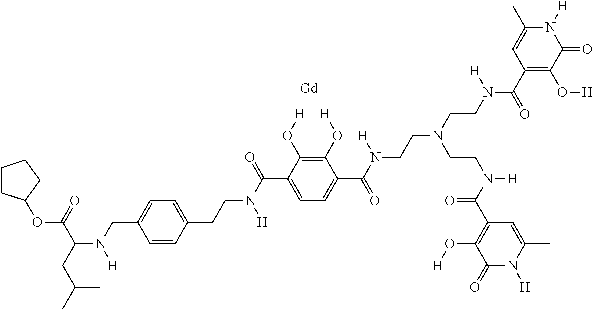 |
| 12 | 9 | X-Ray | 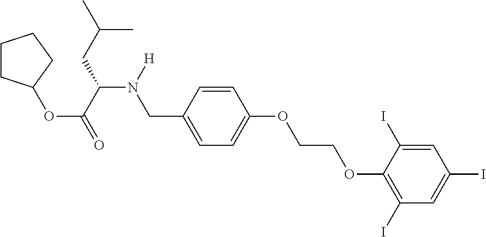 [SMILES: CC(C)C[C@H](NCc1ccc(OCCOc2c(I)cc(I)cc2I)cc1)C(=O)OC1CCCC1] |

*Cu-64 is a radioactive nuclide of copper which has unique decay properties making it useful in imaging. The Cu-64 complex is based upon a known copper chelator (Solvent Extraction. Classical and Novel Approaches, Vladimir S. Kislik. Also, J. lnorg. Nucl. Chem, Vol 42, pp 431-439).

A similar approach can also be used for the other imaging agents identified in Table 1.

It can be seen from the above that the position of attachment of the imaging agent to the alpha mono- or di-substituted amino acid ester can vary widely and in general a suitable point of attachment can be selected by the person skilled in the art. The attachment point of the amino acid ester moiety should be selected so as to preserve the function of the imaging agent and the hydrolysability of the alpha amino acid ester group. The imaging agents of the invention can be synthesised by separate production of the amino acid section and the imaging agent, and by conjugation of the two parts, for example using standard addition chemistry. Alternatively, the imaging agent of the invention may be synthesised from alternative starting materials. Exemplary syntheses are given in the Examples section below.

It will further be apparent from the above Tables 1 and 2 that in formulae (IA′) and (IB′) above, the imaging agent radical Im may be selected from:

imaging agents for use in PET, for example ¹⁸F, ¹¹C, ¹³N and ⁶⁴Cu, including ⁶⁴Cu complexes, for example

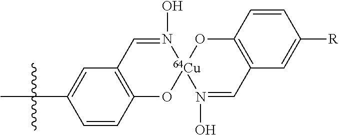

wherein R is H or a substituent e.g. a C₁-C₆ alkyl group;
imaging agents for use in SPECT for example ⁹⁹Tc, ¹²³I and ²⁰¹Tl and radicals containing ⁹⁹Tc, ¹²³I or ²⁰¹Tl, for example

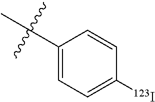 [SMILES: *c1ccc([123I])cc1]

fluorophores, for example

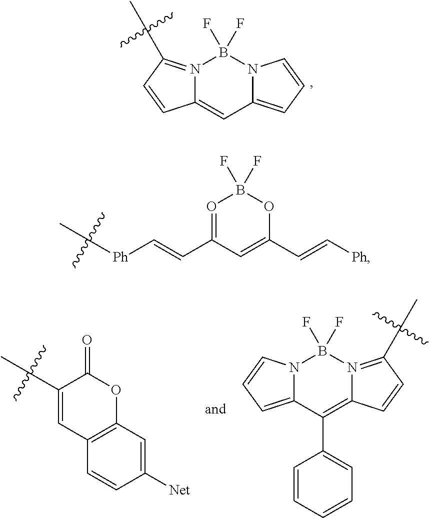

and derivatives thereof including substituted radicals thereof;
MRI contrast agents including Gd³⁺ complexes, for example

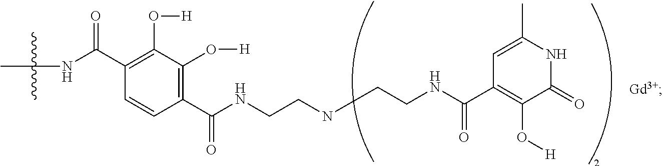

X-ray imaging agents such as iodine-containing radicals, for example 2,4,6-iodophenyl compounds, for example

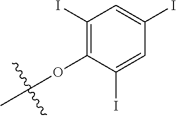 [SMILES: *Oc1c(I)cc(I)cc1I]

The invention is now described, by way of example only, with reference to the Figures of which:

FIG. 1 shows the absolute expression of hCE-1, hCE-2, and hCE-3 in monocytic and non-monocytic cells lines; and

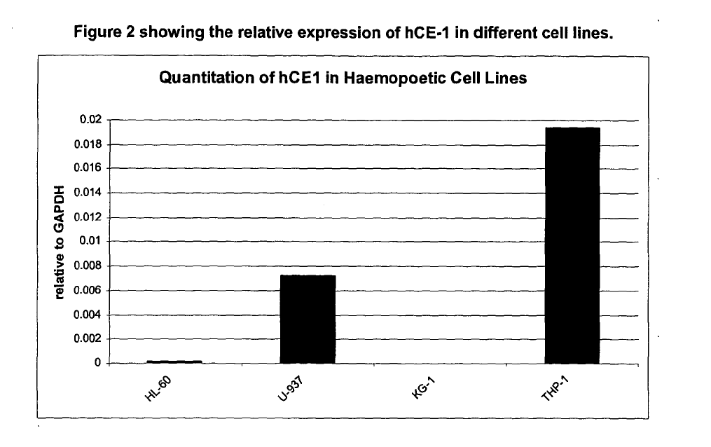

FIG. 2 shows the relative expression of hCE-1 in THP-1, HL-60 and KG-1 cell lines; and

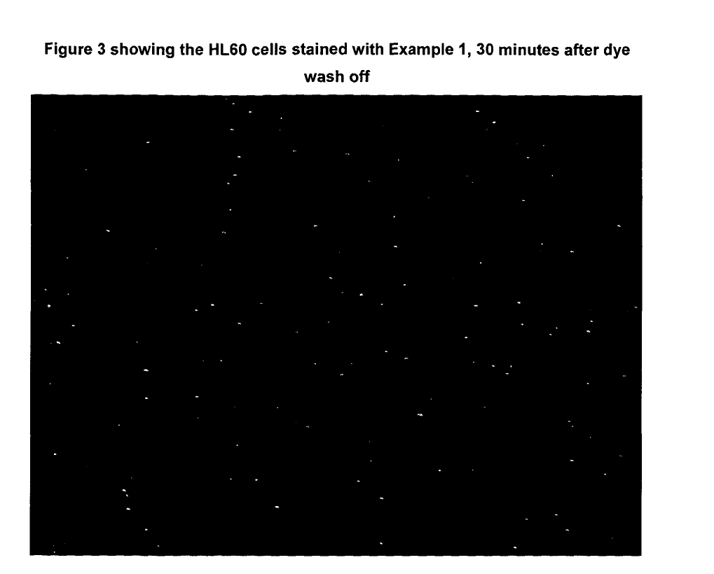

FIG. 3 shows HL60 cells (containing insignificant quantities of hCE-1) stained with an imaging agent according to the invention (Example 1), 30 minutes after dye wash off; and

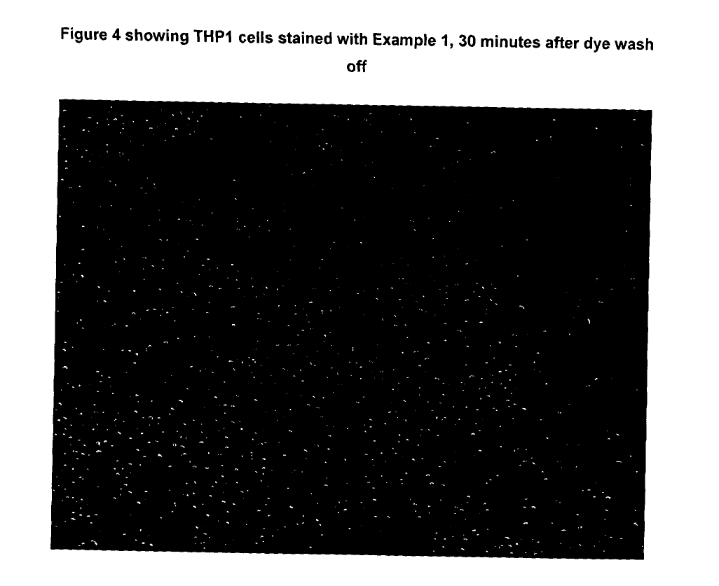

FIG. 4 shows THP1 cells (containing significant quantities of hCE-1) stained with an imaging agent according to the invention (Example 1), 30 minutes after dye wash off; and

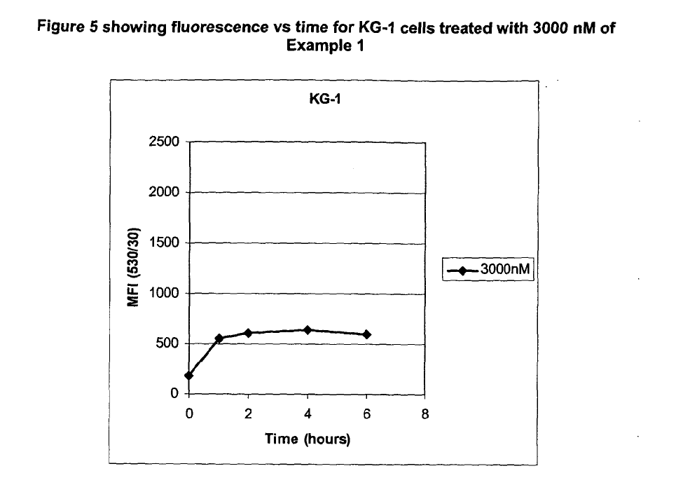

FIG. 5 shows graphically the low increase in fluorescence following treatment of KG-1 cells (containing insignificant quantities of hCE-1) with an imaging agent according to the invention (Example 1); and

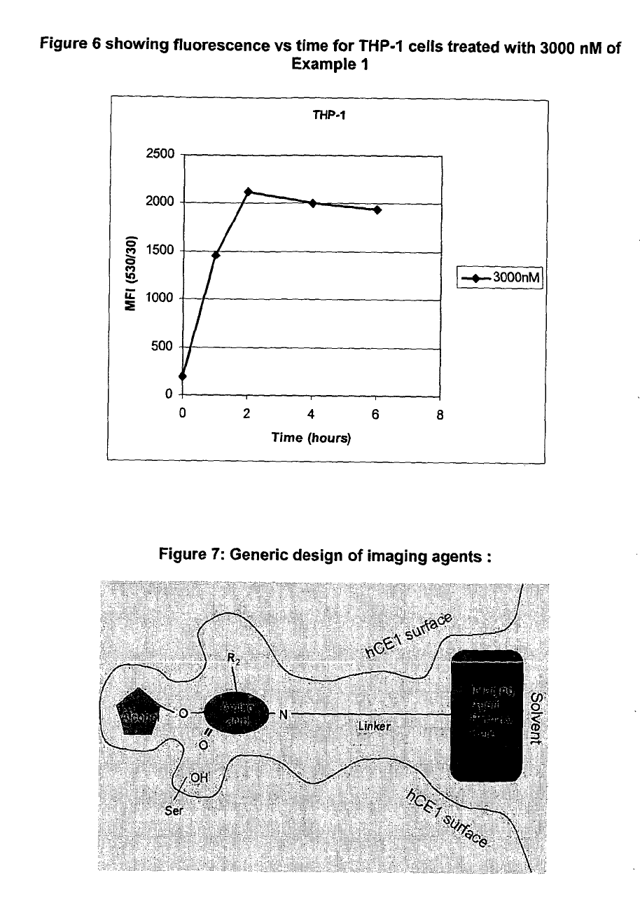

FIG. 6 shows graphically the high increase in fluorescence following treatment of THP-1 cells (containing significant quantities of hCE-1) with an imaging agent according to the invention (Example 1).

FIG. 7 shows schematically the docking of the imaging agent of the invention to hCE-1.

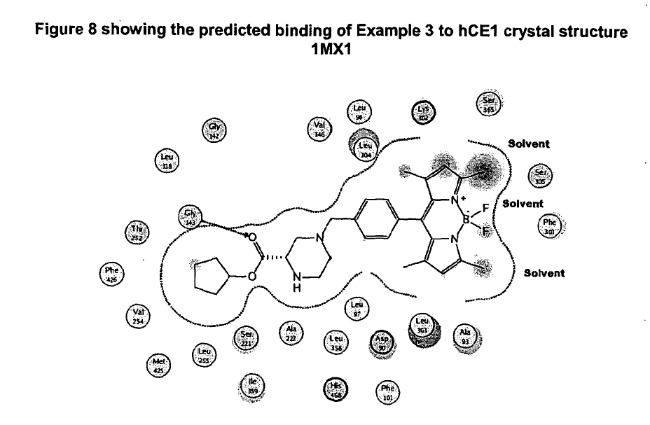

FIG. 8 shows the compound of Example 3 and its predicted binding to hCE1 crystal structure 1XM1.

It can be seen that treatment with an imaging agent according to the invention enables cells expressing significant quantities of hCE-1 (FIG. 4) to be more easily distinguished from the background compared to those cells not significantly expressing hCE-1 (FIG. 3). In this way, the imaging agent conjugate enables selective imaging of cells expressing significant quantities of hCE-1 including monocytic cells, such as macrophages. In other words, the claimed imaging agent conjugates selectively light up monocytic cells compared to non-monocytic cells.

It can be seen from the graphs shown in FIGS. 5 and 6 that following treatment with an imaging agent according to the invention, THP-1 monocytic cells expressing significant quantities of hCE-1 (FIG. 6) exhibit a much higher level of fluorescence compared to KG-1 non-monocytic cells not significantly expressing hCE-1 (FIG. 5). As explained above, the greater fluorescence observed in monocytic cells is due to the selective hydrolysis of the amino acid ester of the conjugate by hCE-1.

The preferred attachment point(s) of the amino acid ester radical to the imaging agent can be considered using docking studies of the proposed imaging agent/amino acid ester conjugate in the X-ray crystal structure of hCE-1 to ensure a good fit of the ester into the active site of the enzyme, as depicted in FIG. 7. The site of attachment of the linker should not interfere with the main imaging core structure.

The invention will now be described in detail with reference to Examples 1 to 3 which are specific embodiments of the invention.

### Example 1

### Cyclopentyl (2S)-[(4-{3-[(7-nitro-2,1,3-benzoxadiazol-4-yl)amino]propoxy}benzyl)amino](phenyl)ethanoate

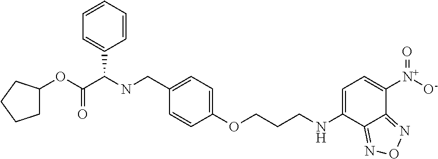 [SMILES: O=C(OC1CCCC1)[C@@H](NCc1ccc(OCCCNc2ccc([N+](=O)[O-])c3nonc23)cc1)c1ccccc1]

The imaging agent Example 1 is a covalent conjugate of a fluorescent imaging agent and an alpha-substituted amino acid ester. Upon entry into cells, the amino acid ester motif is selectively hydrolysed by hCE-1 to the corresponding acid, having low cell permeability, thus causing the hydrolysed conjugate to selectively accumulate within cells having a significant expression of hCE-1, such as monocytic cells, for example macrophages.

### Preparation of Example 1

The imaging agents of the invention, in particular Example 1, may be prepared, by the methods described below.

### Synthesis

There are multiple synthetic strategies for the synthesis of the imaging agents with which the present invention is concerned, but all rely on known chemistry, known to the synthetic organic chemist. Thus, the imaging agents can be synthesised according to procedures described in the standard literature and are well-known to the one skilled in the art. Typical literature sources are “Advanced organic chemistry”, 4M Edition (Wiley), J March; “Comprehensive Organic Transformation”, 2^{nd} Edition (Wiley), R. C. Larock; “Handbook of Heterocyclic Chemistry”, 2^{nd} Edition (Pergamon), A. R. Katritzky; review articles such as found in “Synthesis”, “Acc. Chem. Res.”, “Chem. Rev”, or primary literature sources identified by standard literature searches online or from secondary sources such as “Chemical Abstracts” or “Beilstein”. The synthetic routes used in the preparation of Example 1, and intermediates thereof, may be adapted for the preparation of analogous compounds.

### ABBREVIATIONS

MeOH=methanol
EtOH=ethanol
EtOAc=ethyl acetate
Boc=tert-butoxycarbonyl
BF₃.OEt₂=boron trifluoride diethyl etherate
DCM=dichloromethane
DCE=1,2-dichloroethane
DMAP=dimethylaminopyridine
TFA=trifluoroacetic acid
THF=tetrahydrofuran
NaHCO₃=sodium hydrogen carbonate
HCl=hydrochloric acid
aq=aqueous solution
sat=saturated
STAB=sodium triacetoxyborohydride
N₂=nitrogen
Na₂SO₄=sodium sulphate
Et₃N=triethylamine
MgSO₄=magnesium sulfate
EDC=N-(3-Dimethylaminopropyl)-N′-ethylcarbodiimide hydrochloride
TLC=thin layer chromatography
LCMS=liquid chromatography/mass spectrometry
mL=millilitre(s)
g=gram(s)
mg=milligram(s)
mol=mole(s)
mmol=millimole(s)
HPLC=high performance liquid chromatography
NMR=nuclear magnetic resonance
r.t=room temperature
hr(s)=hour(s)

Commercially available reagents and solvents (HPLC grade) were used without further purification. Solvents were removed using a Buchi rotary evaporator. Microwave irradiation was carried out using a Biotage Initiator™ Eight microwave synthetiser. Purification of compounds by flash chromatography column was performed using silica gel, particle size 40-63μ μm (230-400 mesh) obtained from Fluorochem. Reverse phase column chromatography was performed using a pre-column on Merck liChroprep RP-18 (40-60 μm) before purification on a CombiFlash Companion (Teledyne Isco, Nebraska, USA) using RediSep Rf C18 columns (Presearch, Basingstoke, UK). Purification of compounds by preparative HPLC was performed on Gilson systems using reverse phase Axia™ prep Luna C18 columns (10 μmu, 100×21.2 mm), gradient 0-100% B (A=water/0.05% TFA, B=acetonitrile/0.05% TFA) over 10 min, flow=25 ml/min, UV detection at 254 nm.

¹H NMR spectra were recorded on a Bruker 300 MHz AV spectrometer in deuterated solvents. Chemical shifts (δ) are in parts per million. Thin-layer chromatography (TLC) analysis was performed with Kieselgel 60 F₂₅₄ (Merck) plates and visualized using UV light. Analytical HPLC/MS was performed on an Agilent HP1100 LC system using reverse phase Luna C18 columns (3μ μm, 50×4.6 mm), gradient 5-95% B (A=water/0.1% Formic acid, B=acetonitrile/0.1% Formic acid) over 2.25 min, flow=2.25 ml/min. UV spectra were recorded at 220 and 254 nm using a G1315B DAD detector. Mass spectra were obtained over the range m/z 150 to 800 on a LC/MSD SL G1956B detector. Data were integrated and reported using ChemStation and ChemStation Data Browser softwares.

### Scheme 1—Methodology for the Preparation of Example 1

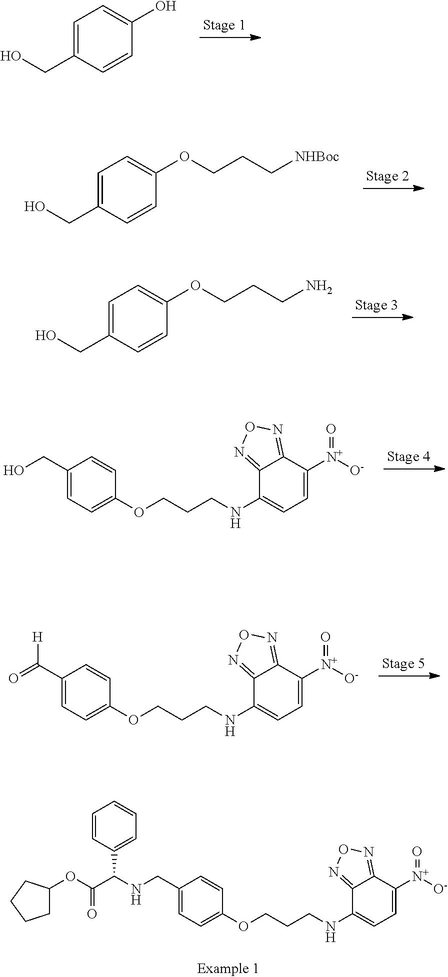 [SMILES: OCc1ccc(O)cc1 ; CC(C)(C)OC(=O)NCCCOc1ccc(CO)cc1 ; NCCCOc1ccc(CO)cc1 ; O=[N+]([O-])c1ccc(NCCCOc2ccc(CO)cc2)c2nonc12 ; O=Cc1ccc(OCCCNc2ccc([N+](=O)[O-])c3nonc23)cc1 ; O=C(OC1CCCC1)[C@@H](NCc1ccc(OCCCNc2ccc([N+](=O)[O-])c3nonc23)cc1)c1ccccc1]

### Step 1. tert-Butyl {3-[4-(hydroxymethyl)phenoxy]propyl}carbamate

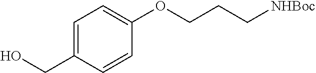 [SMILES: CC(C)(C)OC(=O)NCCCOc1ccc(CO)cc1]

To a solution of 4-hydroxybenzyl bromide (0.1 g, 0.8 mmol) and tert-butyl (3-bromopropyl)carbamate (0.29 g, 1.2 mmol) in DMF (5 mL) was added potassium carbonate (0.44 g, 3.2 mmol). The reaction was heated at 70° C. overnight. TLC indicated only 30% completion and so the reaction was heated overnight again. The reaction was diluted with ethyl acetate (10 mL) and washed with water (10 mL). The aqueous layer was washed with ethyl acetate (1×5 mL) and the combined organic layers dried (Na₂SO₄). The solvent was removed under reduced pressure and the residue purified by preparative TLC (silica gel, 50% ethyl acetate in heptanes) to give the product (120 mg) as a viscous oil.

LCMS: m/z 282 [M+H]⁺.

### Step 2. [4-(3-Aminopropoxy)phenyl]methanol Trifluoroacetate

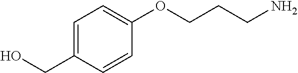 [SMILES: NCCCOc1ccc(CO)cc1]

tert-Butyl {3-[4-(hydroxymethyl)phenoxy]propyl}carbamate (120 mg) was dissolved in 2% TFA in DCM and allowed to stand at r.t. for 20 mins. The solvent was removed under reduced pressure to give [4-(3-aminopropoxy)phenyl]methanol trifluoroacetate (135 mg),

LCMS: m/z 182 [M+H]⁺.

### Step 3. (4-{3-[(7-nitro-2,1,3-benzoxadiazol-4-yl)amino]propoxy}phenyl)methanol

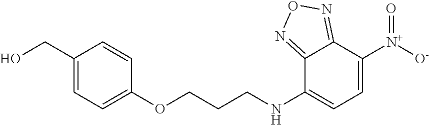 [SMILES: O=[N+]([O-])c1ccc(NCCCOc2ccc(CO)cc2)c2nonc12]

To a stirred solution of [4-(3-aminopropoxy)phenyl]methanol trifluoroacetate (135 mg, 0.47 mmol) in EtOH (2 mL) was added potassium carbonate (126 mg, 0.91 mmol). To this solution was added dropwise a solution of 4-chloro-7-nitro-2,1,3-benzoxadiazole (91 mg, 0.47 mmol) in EtOH (3 mL) at r.t. for 2 hrs. The solvent was evaporated and the residue purified by preparative TLC (silica gel, 50% ethyl acetate in heptanes) to give (4-{3-[(7-nitro-2,1,3-benzoxadiazol-4-yl)amino]propoxy}phenyl)methanol (20 mg) as a bright yellow solid. LCMS: m/z 345 [M+H]⁺.

### Step 4. 4-{3-[(7-nitro-2,1,3-benzoxadiazol-4-yl)amino]propoxy}benzaldehyde

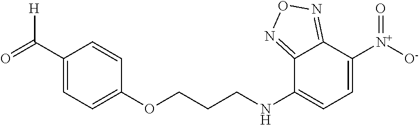 [SMILES: O=Cc1ccc(OCCCNc2ccc([N+](=O)[O-])c3nonc23)cc1]

To a solution of 4-{3-[(7-nitro-2,1,3-benzoxadiazol-4-yl)amino]propoxy}phenyl)methanol (20 mg, 5.8 mmol) in CH₂Cl₂ (0.5 mL) was added manganese dioxide (25 mg, 0.29 mmol) and the reaction stirred at r.t. overnight. Additional manganese dioxide (12 mg) was added and stirring continued for 5 days. The reaction was filtered through a pad of Celite, which was thoroughly washed with DCM. The combined filtrates were evaporated and the residue used in the next step without purification.

### Step 5. Cyclopentyl (2S)-[(4-{3-[(7-nitro-2,1,3-benzoxadiazol-4-yl)amino]propoxy}benzyl)amino](phenyl)ethanoate

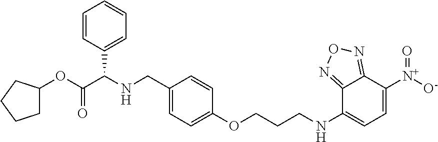 [SMILES: O=C(OC1CCCC1)[C@@H](NCc1ccc(OCCCNc2ccc([N+](=O)[O-])c3nonc23)cc1)c1ccccc1]

To a solution of 4-{3-[(7-nitro-2,1,3-benzoxadiazol-4-yl)amino]propoxy}benzaldehyde (20 mg, 0.01 mmol) and cyclopentyl L-phenylglycinate tosylate* (33 mg, 0.01 mmol) was added potassium carbonate (11 mg, 0.01 mmol), sodium cyanoborohydride (7 mg, 0.011 mmol) and acetic acid (1 drop) and the reaction stirred at r.t. for 12 hours. The reaction was evaporated to dryness and the residue partitioned between EtOAc (10 mL) and water (3 mL) and the organic layer washed with (2×3 mL). The organic layer was dried (Na₂SO₄) and evaporated. The residue was purified by preparative TLC (60% EtOAc in heptanes) to give cyclopentyl (2S)-[(4-{3-[(7-nitro-2,1,3-benzoxadiazol-4-yl)amino]propoxy}benzyl)amino](phenyl)ethanoate (8 mg).

LCMS: m/z 546 [M+H]. ¹H NMR (CD₃OD) δ ppm: 8.39 (1H, d, J=8.0 Hz), 7.31-7.16 (5H, m), 7.08 (1H, d, J=7.0 Hz), 6.82 (1H, d, J=7.0 Hz), 6.31 (1H, d, J=7.0 Hz), 5.48 (1H, s), 5.02 (1H, m), 4.17 (2H, s), 4.03 (2H, m), 3.51 (2H, m), 2.47 (2H, m), 2.14 (2H, m), 1.78-1.26 (10H, m).

The synthesis of cyclopentyl L-phenylglycinate tosylate is described below.

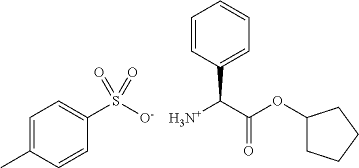 [SMILES: Cc1ccc(S(=O)(=O)[O-])cc1 ; [NH3+][C@H](C(=O)OC1CCCC1)c1ccccc1]

To a slurry of (S)-phenylglycine (5 g, 33.1 mmol) in cyclohexane (150 mL) was added cyclopentanol (29.84 ml, 331 mmol) and p-toluene sulfonic acid (6.92 g, 36.4 mmol). The reaction was fitted with a Dean-Stark receiver and heated to 135° C. for complete dissolution. After 12 hrs, the reaction was cooled to r.t leading to the precipitation of a white solid. The solid was filtered and washed with EtOAc before drying under reduced pressure to give the required product as a white powder (11.01 g, 85%). ¹H NMR (300 MHz, d₆-DMSO) δ; 8.82 (2H, br s), 8.73 (1H, br s), 7.47 (7H, m), 7.11 (2H, d), 5.25 (1H, br s), 5.18 (1H, m), 2.29 (3H, s), 1.87-1.36 (8H, m).

The following building blocks were employed in the synthesis of Examples 2 and 3 described herein:

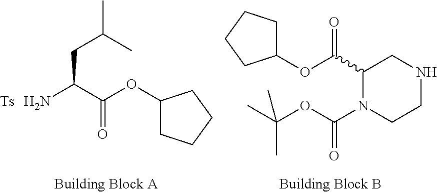

### Building Block A—Cyclopentyl L-Leucinate was Prepared Using the Methodology Described Below:

To a slurry of L-Leucine (5 g, 30.5 mmol) in cyclohexane (150 mL) were added cyclopentanol (27.5 mL, 305 mmol) and p-toluene sulfonic acid (6.33 g, 33.3 mmol). The reaction was fitted with a Dean-Stark receiver and heated to 135° C. for complete dissolution. This temperature was maintained for a period of 12 hours after which time the reaction was complete. The reaction was cooled to r.t with precipitation of a white solid. The solid was filtered and washed with EtOAc before drying under reduced pressure. The required product was isolated as the tosylate salt (10.88 g, 85%).

LCMS: m/z=200 [M+H]⁺. ¹H NMR (300 MHz, CD₃OD) δ: 1.01 (6H, t, J=5.8 Hz), 1.54-2.03 (11H, m), 2.39 (3H, s), 3.96 (1H, t, J=6.5 Hz), 5.26-5.36 (1H, m), 7.25 (2H, d, J=7.9 Hz), 7.72 (2H, d, J=8.3 Hz).

### Building Block B—1-tert-Butyl 2-cyclopentyl piperazine-1,2-dicarboxylate

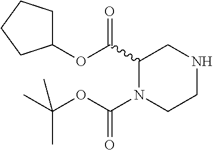 [SMILES: CC(C)(C)OC(=O)N1CCNCC1C(=O)OC1CCCC1]

1-tert-Butyl 2-cyclopentyl piperazine-1,2-dicarboxylate was synthesised using the route shown in Scheme 2.

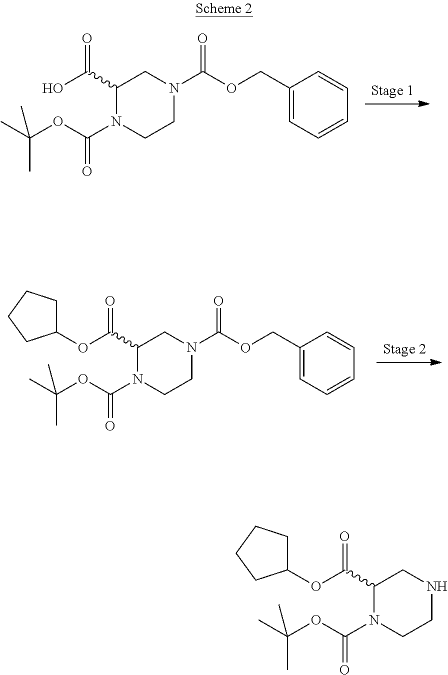 [SMILES: CC(C)(C)OC(=O)N1CCN(C(=O)OCc2ccccc2)CC1C(=O)O ; CC(C)(C)OC(=O)N1CCN(C(=O)OCc2ccccc2)CC1C(=O)OC1CCCC1 ; CC(C)(C)OC(=O)N1CCNCC1C(=O)OC1CCCC1]

### Stage 1. 4-Benzyl 1-tert-butyl 2-cyclopentyl piperazine-1,2,4-tricarboxylate

4-[(Benzyloxy)carbonyl]-1-(tert-butoxycarbonyl)piperazine-2-carboxylic acid (9.96 g, 27 mmol) was dissolved in DCM (50 mL) and cooled to 0° C. Cyclopentanol (7.4 mL, 82 mmol), EDC (7.86 g, 41 mmol) and DMAP (0.33 g, 2.7 mmol) were then added and the reaction allowed to warm to r.t, and stirred for 24 hrs. Water (100 mL) and DCM (50 mL) were then added and the layers separated. The aqueous layer was re-extracted with DCM (2×50 mL) and the combined organic layers were then dried over MgSO₄ and concentrated in vacuo. Purification by column chromatography (40% EtOAc/Heptane) afforded the title compound as a colourless oil (10.4 g, 88%).

LC/MS: m/z 455 [M+Na]⁺.

### Stage 2. 1-tert-Butyl 2-cyclopentyl piperazine-1,2-dicarboxylate

4-Benzyl 1-tert-butyl 2-cyclopentyl piperazine-1,2,4-tricarboxylate (10.4 g) was dissolved in ethyl acetate (100 mL), and stirred with 10% palladium on carbon (2 g, 20% w/w) under hydrogen at atmospheric pressure for 18 hrs. The reaction mixture was then filtered through Celite® and the solvent removed in vacuo to give the title compound as a white solid (6.62 g, 92%).

¹H NMR (300 MHz, CDCl₃): 5.27 (1H, m), 4.50 (1H, m), 3.81 (1H, m), 3.50 (1H, m), 2.82-3.2 (3H, m), 2.72 (1H, m), 1.55-1.95 (8H, br m), 1.45 (9H, s).

### Example 2

### 1,3,5,7-Tetramethyl-8-(4-cyclopentyl N-benzyl-L-leucinate)-4,4′-difluoroborodiazaindacene

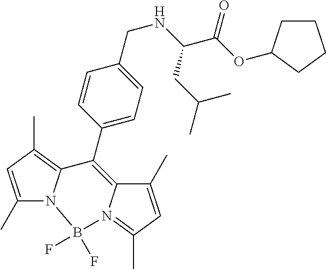

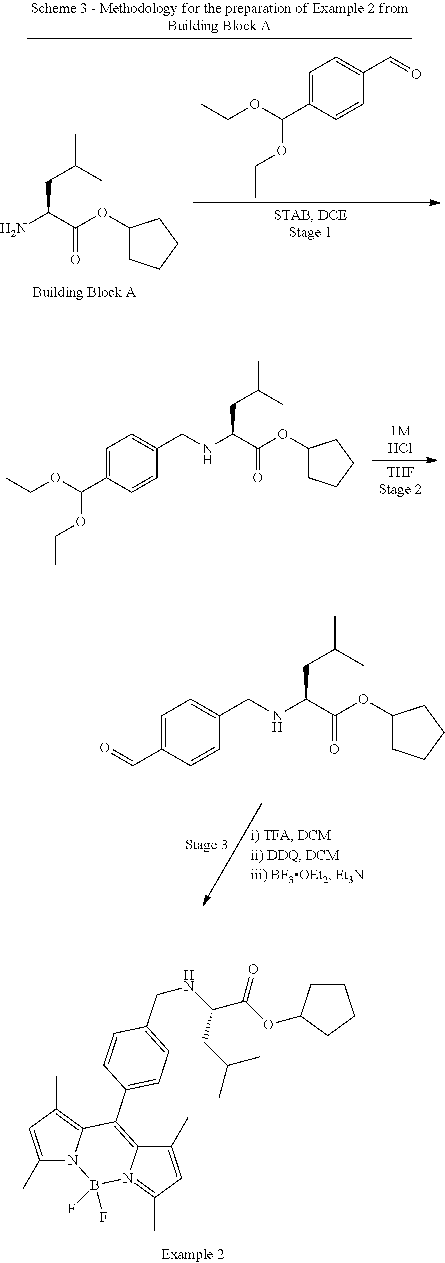

### Stage 1—Cyclopentyl N-[4-(diethoxymethyl)benzyl]-L-leucinate

To Building Block A (5.35 g, 14.4 mmol) in DCE (20 mL) was added terephthaldehyde mono-diethyl acetal (2 g, 9.6 mmol). The reaction mixture was stirred at room temperature for 1 hour and then STAB (4.07 g, 19.2 mmol) was added portionwise and stirred at room temperature for 18 hrs. DCM (100 mL) was added and the reaction mixture washed with sat. NaHCO₃ (2×100 mL), dried (MgSO₄) and concentrated under reduced pressure. Purification by flash column chromatography (10%-25% EtOAc/Heptane) afforded the desired product as a colourless oil (1.85 g, 49% yield).

LC/MS: m/z 392 [M+H]⁺.

### Stage 2—Cyclopentyl N-(4-formylbenzyl)-L-leucinate

To cyclopentyl N-[4-(diethoxymethyl)benzyl]-L-leucinate (1.85 g) in THF (10 mL) was added 1M HCl (10 mL). The reaction mixture was stirred at room temperature for 18 hrs for complete reaction. The THF was removed under reduced pressure and EtOAc (50 mL) added, washing with sat. NaHCO₃ (100 mL), dried (MgSO₄) and concentrated under reduced pressure to afford the desired product as a colourless oil, which was taken forward without further purification. (1.18 g, 79% yield).

LC/MS: m/z 318 [M+H]⁺.

¹H NMR (300 MHz, CDCl₃) δ: 10.0 (1H, s), 7.84 (2H, d, J=8.1 Hz), 7.51 (2H, d, J=8.1 Hz), 5.25 (1H, m), 3.91 (1H, d J=13.8 Hz), 3.68 (1H, d, J=13.8 Hz), 3.2 (1H, t, J=7.2 Hz), 1.95-1.55 (9H, bm), 1.45 (2H, t, J=7.5 Hz), 0.93 (3H, d J=6.9 Hz), 0.88 (3H, d, J=6.9 Hz).

### Stage 3—1,3,5,7-Tetramethyl-8-(4-cyclopentyl N-benzyl-L-leucinate)-4,4′-difluoroborodiazaindacene

Cyclopentyl N-(4-formylbenzyl)-L-leucinate (1.18 g, 3.72 mmol) was dissolved in DCM (250 mL) and 2,4-dimethylpyrrole (707 mg, 7.44 mmol) added. 3 drops of TFA was added and stirred at room temperature for 15 hrs. An additional 2 drops of TFA was added and stirred for 2 hrs for complete reaction. Tetrachlorobenzoquinone (915 mg, 3.72 mmol) in DCM (150 mL) was then added, stirring for 30 minutes before the addition of Et₃N (12 mL) and BF₃.OEt₂ (12 mL). After stirring for another 18 hrs, the crude reaction mixture was washed with water (2×100 mL), dried (MgSO₄) and concentrated under reduced pressure. Purification by column chromatography (0%-5% MeOH/DCM) afforded the required product which was slurried in heptane (150 mL). The heptane layer was collected, evaporated under reduced pressure to give Example 2 as a dark red gum (184 mg, 9%).

¹H NMR (300 MHz, CDCl₃) 5 ppm: 7.4 (2H, d, J=8.4 Hz), 7.15 (2H, d, J=8.4 Hz), 5.9 (2H, s), 5.2 (1H, m), 3.85 (1H, d, J=13.2 Hz), 3.6 (1H, d, J=13.2 Hz), 3.15 (1H, t, J=7.2 Hz), 2.48 (6H, s), 1.75-1.9 (3H, m), 1.51-1.74 (8H, m), 1.4 (1H, t, J=7.0 Hz), 1.31 (6H, s), 0.85 (3H, d, J=6.5 Hz), 0.79 (3H, d, J=6.4 Hz).

LCMS: m/z 537 [M+H]⁺

### Example 3

### 1,3,5,7-Tetramethyl-8-(4-cyclopentyl 4-benzylpiperazine-2-carboxylate)-4,4′-difluoroborodiazaindacene

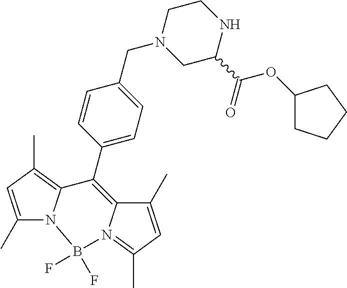

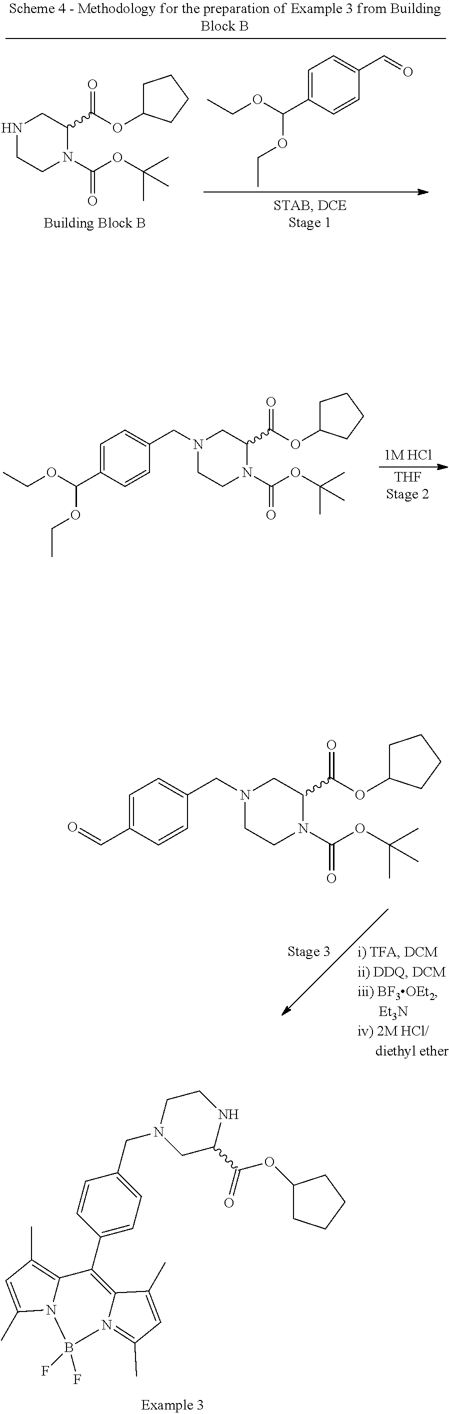

### Stage 1—1-tert-Butyl 2-cyclopentyl 4-[4-(diethoxymethyl)benzyl]piperazine-1,2-dicarboxylate was prepared from building block B as follows

To Building Block B (479 mg, 1.6 mmol) in DCM (5 mL) was added terephthaldehyde mono-diethyl acetal (222 mg, 1.07 mmol). The reaction mixture was stirred at room temperature for 1 hr and then STAB (453 mg, 2.14 mmol) was added portionwise and stirred at room temperature for 18 hours. DCM (100 mL) was added and the reaction mixture washed with sat. NaHCO₃ (2×50 mL), dried (MgSO₄) and concentrated under reduced pressure to afford the required product as a clear oil which was taken forward without further purification (606 mg, >100% yield).

LC/MS: m/z 491 [M+H]⁺.

### Stage 2—1-tert-Butyl 2-cyclopentyl 4-(4-formylbenzyl)piperazine-1,2-dicarboxylate

To 1-tert-butyl 2-cyclopentyl 4-[4-(diethoxymethyl)benzyl]piperazine-1,2-dicarboxylate (606 mg) in THF (6 mL) was added 1M HCl (6 mL). The reaction mixture was stirred at room temperature for 18 hrs for complete reaction. The reaction mixture was diluted with EtOAc (50 mL), washing with sat. NaHCO₃ (2×50 mL), dried (MgSO₄) and concentrated under reduced pressure to afford the desired product as a colourless oil, which was taken forward without further purification. (440 mg, 99% yield).

LC/MS: m/z 417 [M+H]⁺.

### Stage 3—1,3,5,7-Tetramethyl-8-(4-cyclopentyl 4-benzylpiperazine-2-carboxylate)-4,4′-difluoroborodiazaindacene

1-tert-Butyl 2-cyclopentyl 4-(4-formylbenzyl)piperazine-1,2-dicarboxylate (440 mg, 1.06 mmol) was dissolved in DCM (90 mL) and 2,4-dimethylpyrrole (200 mg, 2.12 mmol) added. 3 drops of TFA was added and stirred at room temperature for 15 hrs for complete reaction. Tetrachlorobenzoquinone (260 mg, 1.06 mmol) in DCM (60 mL) was then added, stirring for 15 minutes before the addition of Et₃N (5 mL) and BF₃.OEt₂ (5 mL). After stirring for another 18 hrs, the crude reaction mixture was washed with water (2×100 mL), dried (MgSO₄) and concentrated under reduced pressure. Purification by column chromatography (0%-5% MeOH/DCM) afforded material which was further slurried in heptane (100 mL). The heptane layer was collected and evaporated under reduced pressure. LCMS data showed there was a mixture of Boc-protected and unprotected products.

The 112 mg of crude material was dissolved in 2M HCl in diethyl ether (50 mL) and stirred at room temperature for 18 hrs. The reaction mixture was concentrated under reduced pressure and purified by column chromatography (0%-5% MeOH/DCM) to afford the desired product as an orange oil (5.6 mg, 3%).

LC/MS: m/z 535 [M+H]⁺.

¹H NMR (300 MHz, CDCl₃) δ: 7.37 (2H, d, J=8.1 Hz), 7.15 (2H, d, J=8.1 Hz), 5.91 (2H, s), 5.14 (1H, m), 3.55 (2H, s), 3.49 (1H, m), 3.05 (1H, m), 2.80 (2H, m), 2.48 (6H, s), 2.35 (1H, m), 2.27-2.00 (3H, bm), 1.80 (2H, m), 1.69-1.45 (6H, bm), 1.30 (6H, s).

FIG. 8 shows the predicted binding of Example 3 to hCE1 crystal structure 1MX1 from the Protein Data Bank. The ester is close to Serine 221, enabling ester hydrolysis to occur. Also, the solvent exposed nature of the imaging core means there is little interference of the core with the binding of the amino acid ester to the esterase.

### Biological Assays

### Broken Cell Carboxylesterase Assay

Compounds can be assessed for selective hydrolysis in monocytic cells such as macrophages in a broken cell hydrolysis assay (described below). When this assay is performed on monocytic cells (such as U937 cells) hydrolysis rates typically exceed several thousand pg/ml/min. However, this same experiment carried out with non-monocytic cells (such as HCT116 and HuT78 cells) gives hydrolysis rates that are typically less than 100 pg/ml/min.

### Preparation of Cell Extract

U937 (monocytic or HCT 116 (non-monocytic) tumour cells (˜10⁹) were washed in 4 volumes of Dulbeccos PBS (˜1 litre) and pelleted at 525 g for 10 min at 4° C. This was repeated twice and the final cell pellet was resuspended in 35 ml of cold homogenising buffer (Trizma 10 mM, NaCl 130 mM, CaCl₂ 0.5 mM pH 7.0 at 25° C.). Homogenates were prepared by nitrogen cavitation (700 psi for 50 min at 4° C.). The homogenate was kept on ice and supplemented with a cocktail of inhibitors at final concentrations of:
Leupeptin 1 μM
Aprotinin 0.1 μM
E64 8 μM
Pepstatin 1.5 μM
Bestatin 162 μM
Chymostatin 33 μM

After clarification of the cell homogenate by centrifugation at 525 g for 10 min, the resulting supernatant was used as a source of esterase activity and was stored at −80° C. until required.

### Measurement of Ester Cleavage

Hydrolysis of esters to the corresponding carboxylic acids can be measured using the cell extract, prepared as above. To this effect cell extract (˜30 μg/total assay volume of 0.5 ml) was incubated at 37° C. in a Tris-HCl 25 mM, 125 mM NaCl buffer, pH 7.5 at 25° C. At zero time the ester (substrate) was then added at a final concentration of 2.5 μM and the samples were incubated at 37° C. for the appropriate time (usually 0 or 80 min). Reactions were stopped by the addition of 3× volumes of acetonitrile. For zero time samples the acetonitrile was added prior to the ester compound. After centrifugation at 12000 g for 5 min, samples were analysed for the ester and its corresponding carboxylic acid at room temperature by LCMS (Sciex API 3000, HP1100 binary pump, CTC PAL). Chromatography was based on an AcCN (75*2.1 mm) column and a mobile phase of 5-95% acetonitrile in water/0.1% formic acid.

As set out herein, typically if a carboxyesterase hydrolyses a free amino acid ester (e.g. a free cyclic amino acid ester) to the parent acid it will also hydrolyse the same ester motif when linked via the linker radical —Y-L-X—(CH₂)_{z}— to an imaging agent. Hence, the broken cell assay described herein can be used to test free amino acid esters to determine whether that amino acid ester is hydrolysable by intracellular carboxyesterase enzymes and the result of this test gives a good indication as to the properties of the conjugated molecule. An ester motif selected in that way may then be re-assayed in the same carboxyesterase assay when conjugated to the inhibitor via the respective linker radicals linker radical to confirm that it is still a carboxyesterase substrate in that background.

Quantification of hCE-1, hCE-2 and hCE-3 Expression in Monocytic and Non-Monocytic Cell Lines

Gene-specific primers were used to PCR-amplify hCE-1, -2 and -3 from human cDNA. PCR products were cloned into a plasmid vector and sequence-verified. They were then serially diluted for use as standard curves in real-time PCR reactions. Total RNA was extracted from various human cell lines and cDNA prepared. To quantitate absolute levels of hCE's in the cell lines, gene expression levels were compared to the cloned PCR product standards in a real-time SYBR Green PCR assay. FIG. 1 shows that hCE-1 is only expressed to a significant amount in a monocytic cell line. The same method was used to look at relative expression levels as shown in FIG. 2. In this case the levels were assessed in comparison to GAPDH expression.

### Staining Experiment (as Shown in FIGS. 3 and 4)

THP1 or HL60 cells were seeded the day before staining at 1×10⁴/ml in full media at a total volume of 50 ml. For HL60, RMPI1640+10% foetal calf serum, 1% glutamine and 1% penicillin/streptomycin was used. For THP1, RMPI1640+10% foetal calf serum, 1% glutamine and 1% penicillin/streptomycin 0.05 mM mercaptoethanol was used. The cells were incubated overnight at 37° C. in a humidified incubator with 5% CO₂.

The cells were then centrifuged and washed with 50 ml PBS at 37° C. once before re-suspending in 2 ml serum-free media. The imaging agent Example 1 was prepared at 50 μM in serum-free media and 2 ml added to the cell suspension (4 ml total volume with 25 μM final concentration), and the resulting mixture plated into a 6 well plate, and incubated at 37° C. for 45 minutes. The wash step was repeated, and the cell pellet re-suspended in 4 ml of full media, and incubated at 37° C. for a further 30 minutes.

0.5 ml of the cell suspension was diluted in 4 ml, and 100 μl of each diluted sample transferred to a 96 well plate to be examined using a fluorescence microscope. Images of the two cell lines were collected at the same magnification using both white light illumination to see the cell density and using a 460-490 nm filter to excite the imaging agent Example 1, or the hydrolysis product of Example 1.

### Measurement of Fluorescence v Time in Monocytic and Non-Monocytic Cell Lines (as Shown in FIGS. 5 and 6)

The degree of fluorescence of monocytic and non-monocytic cell lines following treatment with Example 1 was assessed using flow cytometry. This assessment was performed using a BD Biosciences BD FACSCanto™ Flow Cytometry System.

The cell lines are treated with a 3000 nM solution of Example 1. The resultant mixture is sampled at times 0, 1 hr, 2 hr, 4 hr and 6 hr. At each time point a sample of the cells is harvested by centrifugation, washed and allowed to rest for 30 minutes then washed again and re-suspended in a small volume for analysis by flow cytometry (FACSCanto™ A BD Biosciences) at an excitation of 488 nM blue laser line emission measured in an FITC detector using a 502 nm long pass dichroic and a 530±15 band pass filter. Data is expressed as median fluorescence intensity (MFI) for the cell population in the FITC channel, and plotted against time in a line graph. The unstained cells are set to an MFI of 100.

It can be seen from FIGS. 5 and 6 that 2 hours following treatment with an imaging agent according to the invention, the fluorescence intensity of cells expressing a significant quantity of hCE-1 is least three times greater than that of cells not expressing a significant quantity of hCE-1.

1. An imaging agent for cells which produces an intracellular imaging signal proportional to the amount of hCE-1 in the cells independently of the amount of hCE-2 and/or hCE-3 in the cells, said imaging agent being a covalent conjugate of (a) an imaging agent and (b) an alpha mono- or di-substituted amino acid ester, wherein
(a) is directly linked to (b), or (a) is indirectly linked to (b) by a linker radical, and wherein said direct or indirect linkage is via the amino group of (b), and wherein
the amino group is not directly linked to a carbonyl group, and wherein
the said alpha mono- or di-substituted amino acid ester part is selectively hydrolysable to the corresponding carboxylic acid part by the intracellular carboxylesterase enzyme hCE-1 relative to the intracellular enzymes hCE-2 or hCE-3.

2. An imaging agent according to claim 1 wherein the alpha mono- or di-substituted amino acid ester is indirectly linked to the imaging agent by a radical of formula (IA) or (IB): 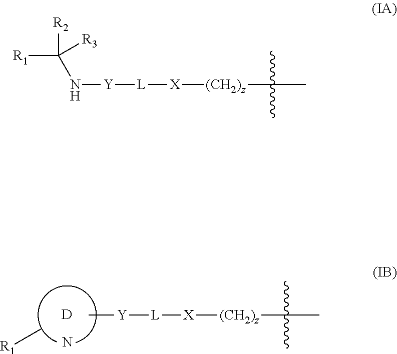
wherein
R₁ is an ester group which is hydrolysable by one or more intracellular carboxylesterase enzymes to a carboxylic acid group;
R₂ is the side chain of a natural or non-natural alpha amino acid;
R₃ is H or R₂;
Y is a bond, —S(═O)₂—, —C(═S)—NR₄—, —C(═NH)NR₄—, —S(═O)₂NR₄—, or —NR₄—C(═O) wherein R₄ is hydrogen or optionally substituted C₁-C₆ alkyl;
L is a divalent radical of formula -(Alk¹)ₘ(Q)ₙ(Alk²)ₚ- wherein
m, n and p are independently 0 or 1,
Q is (i) an optionally substituted divalent mono- or bicyclic carbocyclic or heterocyclic radical having 5-13 ring members, or (ii), in the case where both m and p are 0, a divalent radical of formula —X²-Q¹- or -Q¹-X²— wherein X² is
—O—, —S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent CO₃—C₇cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl;
X represents a bond, —O—, —C(═O)—, —S(═O)₂—, —NR₄C(═O)—, —C(═O)NR₄—, —NR₄C(═O)NR₅—, —NR₄S(═O)₂—, or —S(═O)₂NR₄— wherein R₄ and R₅ are independently hydrogen or optionally substituted C₁-C₆ alkyl;
z is 0 or 1; and
ring D is an optionally substituted 3- to 7-membered heterocyclyl group wherein: R₁ is linked to a ring carbon adjacent to the ring nitrogen shown; and ring D is optionally fused to a second ring comprising a phenyl, 5- to 6-membered heteroaryl, C₃₋₇ carbocyclyl or 5- to 6-membered heterocyclyl group, wherein when the ring D is fused to a second ring comprising a phenyl, 5- to 6-membered heteroaryl, C₃₋₇ carbocyclyl or 5- to 6-membered heterocyclyl group then the linker group —Y-L-X—(CH₂)_{z} may be from a ring atom in ring D or said second ring.

3. An imaging agent according to claim 2 wherein R₃ is H.

4. An imaging agent according to claim 2 wherein the alpha mono- or di-substituted amino acid ester is indirectly linked to the imaging agent by a radical of formula (IA) wherein: R₁ is ester group of formula —(C═O)OR₁₄ wherein R₁₄ is
R₈R₉R₁₀C— wherein
(i) R₈ is hydrogen or optionally substituted (C₁-C₃)alkyl-(Z¹)ₐ—[(C₁-C₃)alkyl]_{b}— or (C₂-C₃)alkenyl-(Z¹)ₐ—[(C₁-C₃)alkyl]_{b}— wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic or bridged monocyclic carbocyclic ring of from 3 to 7 ring atoms or bicyclic or bridged monocyclic carbocyclic ring system of 8 to 10 ring atoms, and R₁₀ is hydrogen;
wherein in cases (i), (ii) and (iii) above, “alkyl” includes fluoroalkyl;
R₂ is selected from:
C₁-C₆ alkyl, phenyl, 2,- 3-, or 4-hydroxyphenyl, 2,- 3-, or 4-methoxyphenyl, 2,- 3-, or 4-pyridylmethyl, benzyl, phenylethyl, 2-, 3-, or 4-hydroxybenzyl, 2,- 3-,
or 4-benzyloxybenzyl, 2,- 3-, or 4-C₁-C₆ alkoxybenzyl, and benzyloxy(C₁-C₆ alkyl)- groups;
the characterising group of a natural w amino acid, in which any functional group may be protected;
groups -[Alk]ₙR₆ where Alk is a (C₁-C₆)alkyl or (C₂-C₆)alkenyl group optionally interrupted by one or more —O—, or —S— atoms or —N(R₇)— groups [where R₇ is a hydrogen atom or a (C₁-C₆)alkyl group], n is 0 or 1, and R₆ is an optionally substituted cycloalkyl or cycloalkenyl group;
a benzyl group substituted in the phenyl ring by a group of formula —OCH₂COR₈ where R₈ is hydroxyl, amino, (C₁-C₆)alkoxy, phenyl(C₁-C₆)alkoxy, (C₁-C₆)alkylamino, di((C₁-C₆)alkyl)amino, phenyl(C₁-C₆)alkylamino, the residue of an amino acid or acid halide, ester or amide derivative thereof, said residue being linked via an amide bond, said amino acid being selected from glycine, □ or □ alanine, valine, leucine, isoleucine, phenylalanine, tyrosine, tryptophan, serine, threonine, cysteine, methionine, asparagine, glutamine, lysine, histidine, arginine, glutamic acid, and aspartic acid;
a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; and
a group —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆)alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl;
R₃ is H or R₂;
Y is a bond;
L is a divalent radical of formula -(Alk¹)ₘ(Q)ₙ(Alk²)ₚ- wherein
m, n and p are independently 0 or 1,
Q is 1,4-phenylene;
Alk¹ and Alk² independently represent —CH₂—, —CH₂CH₂— —CH₂CH₂CH₂—, —CH₂CH₂CH₂CH₂—, —CH═CH—, —CH═CHCH₂—, —CH₂CH═CH—, CH₂CH═CHCH₂—, —C≡C—, —C≡CCH₂—, CH₂C≡C—, CH₂C≡CCH₂, —CH₂W—, —CH₂CH₂W—, —WCH₂CH₂W—, —CH₂CH₂WCH₂—, —CH₂CH₂WCH(CH₃)—, —CH₂WCH₂CH₂—, —CH₂WCH₂CH₂WCH₂—, or —WCH₂CH₂— where W is —O—, —S—, —NH—, —N(CH₃)—, or —CH₂CH₂N(CH₂CH₂OH)CH₂—, or Alk¹ and Alk² may independently represent cyclopropyl, cyclopentyl or cyclohexyl radicals;
X represents a bond, —O—, or —NHC(═O)—; and
z is 0 or 1.

5. An imaging agent according to claim 2 wherein the alpha mono- or di-substituted amino acid ester is indirectly linked to the imaging agent by a radical of formula (IA) wherein: R₁ is an ester group COOR₁₄, wherein R₁₄ is selected from methyl, trifluoromethyl, ethyl, n- or iso-propyl, n-, sec- or tert-butyl, neopentyl, cyclohexyl, cyclopentyl, norbornyl, allyl, phenyl, benzyl, 2-, 3- or 4-pyridylmethyl, N-methylpiperidin-4-yl, tetrahydrofuran-3-yl or methoxyethyl;
R₂ is selected from benzyl, phenyl, cyclohexylmethyl, pyridin-3-ylmethyl, tert-butoxymethyl, iso-butyl, sec-butyl, tert-butyl, 1-benzylthio-1-methylethyl, 1-methylthio-1-methylethyl, and 1-mercapto-1-methylethyl, phenylethyl;
R₃ is H or R₂;
the radical —Y-L-X—(CH₂)_{z} is selected from —(CH₂)ᵥ—, —(CH₂)ᵥO—, (CH₂)_{w}O— 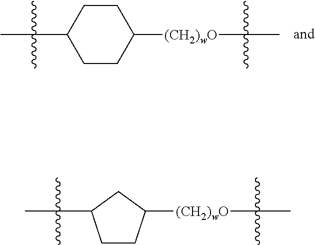
wherein v is 1, 2, 3 or 4 and w is 1, 2 or 3, and from —(CH₂)ₓ-Het²-, —CH₂-Ph-(Het¹)_{yy}-(CH₂)ₓ-(Het²)_{z}- and —CH₂-Ph-CH═CH—, wherein Ph is a 1,4-phenylene group, Het¹ is —O— or —NH—, Het² is —O—, —NH— or —NHC(═O)—, x is 0, 1 or 2, y is 0 or 1 and yy is 0 or 1.

6. An imaging agent according to claim 2 wherein the alpha mono- or di-substituted amino acid ester is indirectly linked to the imaging agent by a radical of formula (IB) wherein: ring D is a saturated, non-fused 5- to 6-membered heterocyclyl group containing, in addition to the nitrogen atom depicted in ring D, none, one or two heteroatoms selected from N, O and S, the ring being substituted with a group R¹ and optionally a group R², wherein R₁ is linked to a ring carbon atom adjacent the ring nitrogen atom shown, and wherein R² is carried on the same carbon atom which carries the group R¹, and wherein the ring D is further unsubstituted or substituted by 1 or 2 groups selected from halogen atoms and C₁₋₄ alkyl, C₁₋₄ alkoxy and hydroxyl groups;
R₁ is ester group of formula —(C═O)OR₁₄ wherein R₁₄ is
R₈R₉R₁₀C— wherein
(i) R₈ is hydrogen or optionally substituted (C₁-C₃)alkyl-(Z)ₐ—[(C₁-C₃)alkyl]_{b}— or (C₂-C₃)alkenyl-(Z¹)ₐ—[(C₁-C₃)alkyl]_{b}— wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₈)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic or bridged monocyclic carbocyclic ring of from 3 to 7 ring atoms or bicyclic or bridged monocyclic carbocyclic ring system of 8 to 10 ring atoms, and R₁₀ is hydrogen;
wherein in cases (i), (ii) and (iii) above, “alkyl” includes fluoroalkyl;
R₂, when present, is selected from:
C₁-C₆ alkyl, phenyl, 2,- 3-, or 4-hydroxyphenyl, 2,- 3-, or 4-methoxyphenyl, 2,- 3-, or 4-pyridylmethyl, benzyl, phenylethyl, 2-, 3-, or 4-hydroxybenzyl, 2,- 3-,
or 4-benzyloxybenzyl, 2,- 3-, or 4-C₁-C₆ alkoxybenzyl, and benzyloxy(C₁-C₆ alkyl)- groups;
the characterising group of a natural □ amino acid, in which any functional group may be protected;
groups -[Alk]ₙR₆ where Alk is a (C₁-C₆)alkyl or (C₂-C₆)alkenyl group optionally interrupted by one or more —O—, or —S— atoms or —N(R₇)— groups [where R₇ is a hydrogen atom or a (C₁-C₆)alkyl group], n is 0 or 1, and R₆ is an optionally substituted cycloalkyl or cycloalkenyl group;
a benzyl group substituted in the phenyl ring by a group of formula —OCH₂COR₈ where R₈ is hydroxyl, amino, (C₁-C₆)alkoxy, phenyl(C₁-C₆)alkoxy, (C₁-C₆)alkylamino, di((C₁-C₆)alkyl)amino, phenyl(C₁-C₆)alkylamino, the residue of an amino acid or acid halide, ester or amide derivative thereof, said residue being linked via an amide bond, said amino acid being selected from glycine, □ or □ alanine, valine, leucine, isoleucine, phenylalanine, tyrosine, tryptophan, serine, threonine, cysteine, methionine, asparagine, glutamine, lysine, histidine, arginine, glutamic acid, and aspartic acid;
a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; and
a group —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆)alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl;
Y is a bond;
L is a divalent radical of formula -(Alk)ₘ(Q)ₙ(Alk²)ₚ- wherein
m, n and p are independently 0 or 1,
Q is 1,4-phenylene;
Alk¹ and Alk² independently represent —CH₂—, —CH₂CH₂— —CH₂CH₂CH₂—, —CH₂CH₂CH₂CH₂—, —CH═CH—, —CH═CHCH₂—, —CH₂CH═CH—, CH₂CH═CHCH₂—, —C≡C—, —C≡CCH₂—, CH₂C≡C—, CH₂C≡CCH₂, —CH₂W—, —CH₂CH₂W—, —WCH₂CH₂W—, —CH₂CH₂WCH₂—, —CH₂CH₂WCH(CH₃)—, —CH₂WCH₂CH₂—, —CH₂WCH₂CH₂WCH₂—, or —WCH₂CH₂— where W is —O—, —S—, —NH—, —N(CH₃)—, or —CH₂CH₂N(CH₂CH₂OH)CH₂—, or Alk¹ and Alk² may independently represent cyclopropyl, cyclopentyl or cyclohexyl radicals;
X represents a bond, —O—, or —NHC(═O)—; and
z is 0 or 1.

7. An imaging agent according to claim 2 wherein the alpha mono- or di-substituted amino acid ester is indirectly linked to the imaging agent by a radical of formula (IB) wherein: ring D is a group selected from 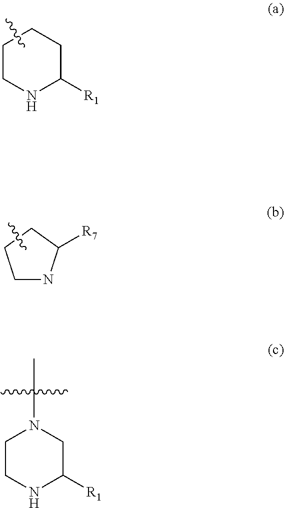
R₁ is an ester group COOR₁₄, wherein R₁₄ is selected from methyl, trifluoromethyl, ethyl, n- or iso-propyl, n-, sec- or tert-butyl, neopentyl, cyclohexyl, cyclopentyl, norbornyl, allyl, phenyl, benzyl, 2-, 3- or 4-pyridylmethyl, N-methylpiperidin-4-yl, tetrahydrofuran-3-yl or methoxyethyl;
the radical —Y-L-X—(CH₂)_{z} is selected from —(CH₂)ᵥ—, —(CH₂)ᵥO—, (CH₂)_{w}O— 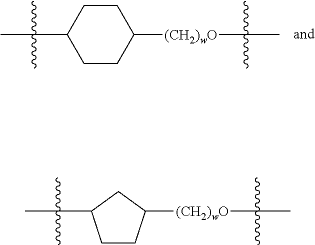
wherein v is 1, 2, 3 or 4 and w is 1, 2 or 3, and from —(CH₂)ₓ-Het²-, —CH₂-Ph-(Het¹)_{yy}—(CH₂)ₓ-(Het²)_{y}- and —CH₂-Ph-CH═CH—, wherein Ph is a 1,4-phenylene group, Het¹ is —O— or —NH—, Het² is —O—, —NH— or —NHC(═O)—, x is 0, 1 or 2, y is 0 or 1 and yy is 0 or 1.

8. An imaging agent according to claim 1 selected from compounds having the formula: 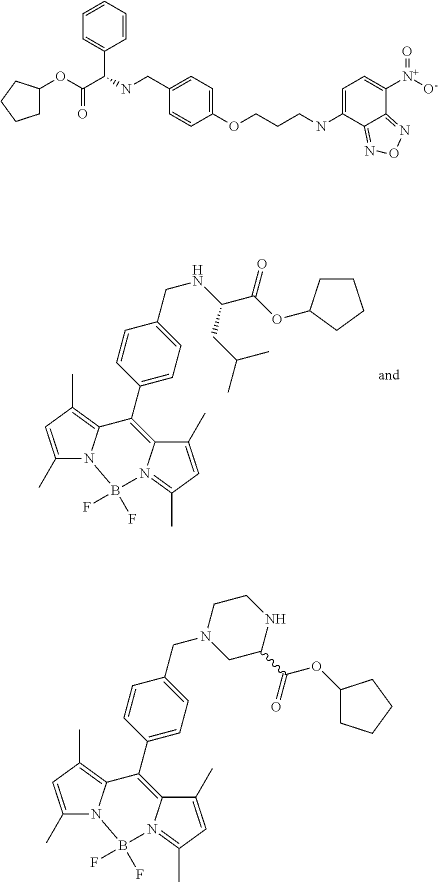
and salts thereof.

9. An imaging agent as claimed in claim 1 for use as an imaging agent for macrophage cells.

10. An imaging method for imaging macrophage cells, comprising carrying out an imaging study on a subject using the imaging agent as defined in claim 1.
